SEQUENCE LISTING (4 sequences)

SEQ ID NO: 1 - DNA, 50 bp, Artificial
  synthetic oligonucleotide
agttgagttagggttagggttagggttagggcaggcggtgtctcgctcgc

SEQ ID NO: 2 - DNA, 50 bp, Artificial
  synthetic oligonucleotide
gcgagcgagacaccgcctgccctaaccctaaccctaaccctaactcaact

SEQ ID NO: 3 - DNA, 50 bp, Artificial
  synthetic oligonucleotide
agttgagttagggttagggttagggttagggcaggcaccacaccattccc

SEQ ID NO: 4 - DNA, 50 bp, Artificial
  synthetic oligonucleotide
gggaatggtgtggtgcctgccctaaccctaaccctaaccctaactcaact

(19) United States
(12) Patent Application Publication
(10) Pub. No.: US 2021/0024455 A1
(43) Pub. Date: Jan. 28, 2021

(54) COMPOUND WHICH INHIBITS TELOMERE-BINDING PROTEIN, AND TELOMERE-BINDING PROTEIN INHIBITOR CONTAINING SAME

(71) Applicant: HIROSHIMA UNIVERSITY, Higashihiroshima-shi, Hiroshima (JP)

(72) Inventors: Hidetoshi TAHARA, Hiroshima-shi, Hiroshima (JP); Yoshitomo SHIROMA, Hiroshima-shi, Hiroshima (JP); Kei TAKEDA, Hiroshima-shi, Hiroshima (JP); Michiko SASAKI, Hiroshima-shi, Hiroshima (JP)

(21) Appl. No.: 16/982,517

(22) PCT Filed: Mar. 14, 2019
(86) PCT No.: PCT/JP2019/010480

§ 371 Date: Sep. 18, 2020

(30) Foreign Application Priority Data
Mar. 20, 2018 (JP) 2018-052087

(51) Int. Cl. C07C 217/92; C07C 225/22; C07C 323/37

(52) U.S. Cl. CPC C07C 217/92; C07C 323/37; C07C 225/22

(57) ABSTRACT

The compound according to the present invention is a compound represented by the following chemical formula:

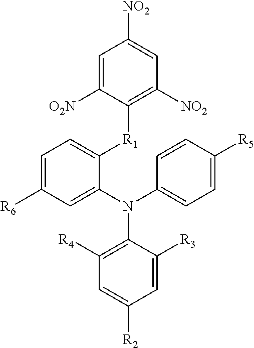

wherein, in the above-described chemical formula, R₁ is oxygen or sulfur, and R₂ to R₆ are each independently selected from hydrogen, an alkyl group having 1 to 6 carbon atoms, an alkoxy group having 1 to 6 carbon atoms, an acyl group having 1 to 6 carbon atoms and a nitro group.

### TECHNICAL FIELD

The present invention relates to a compound that inhibits a telomere-binding protein, and a telomere-binding protein inhibitor containing the same.

### BACKGROUND ART

At the end of human chromosomal DNA, a double-stranded DNA composed of a repeating sequence of 5′-TTAGGG-3′ called a telomere is present. At the extreme end of the telomere, 3′ end is protruded and a single-stranded DNA portion composed of 75 to 300 bases called G tail is formed. Usually, the G tail is in a protected state forming a loop, except when it is accessed by telomerase which is a telomere-extending enzyme or when replicating DNA (see, e.g., Non-Patent Document 1).

It is conventionally known that the double-stranded portion that occupies the majority of telomere becomes shorter each time cell division is repeated, and this is involved in aging of cells. Further, in recent years, POT1 which is a protein that does not bind to the double-stranded telomeric DNA but binds to the G tail, and a protein PIP1 that binds to them, and the like, have been discovered. Furthermore, it has become clear that the G tail of telomere is related to functions that are completely different from the double-stranded portion, for example, a direct signal of cell death and various cell responses as described below.

Telomere has a telomere-binding protein that binds to it, and as the telomere-binding protein, TRF1 (Telomere repeat binding factor) and TRF2 and the like are known, and it has become clear that, in cancer cells, if TRF2 is absent, G-tail loop formation is impossible and G-tail shortening occurs (see, e.g., Non-Patent Document 2). In this case, shortening of the G tail is observed even though there is no change in the total length of telomere, and further, fusion of the chromosome ends is caused. It is known that, even in the case of normal cells, when the function of TRF2 is eliminated in cells, shortening of the G tail occurs, cell growth stops, and aging occurs (see, e.g., Non-Patent Document 2). Also in this case, since the total telomere length does not change, shortening of the G tail is thought to trigger aging.

Not only TRF1 and TRF2 described above, but also various proteins such as ATM, NBS1 and MRN have been found to be required for loop formation of the G tail. Signals sensitive to DNA damage caused by various DNA damaging agents and radiation cause shortening of the G tail even if telomere shortening is not observed. This is clear also from the fact that proteins required for DNA repair (ATM, NBS1, MRN, etc.) are recruited. ATM is a causative gene for vasodilatory diseases, and NBS1 is a causative gene for Nijmegen syndrome. The Nijmegen syndrome is a rare autosomal recessive disorder characterized by high carcinogenicity, immunodeficiency, chromosomal instability and radiosensitivity. Recruitment of these proteins to the G tail shows the relationship with each of the above-described diseases. In fact, if the function of TRF2 working as a G-tail loop paste is inhibited, ATM-dependent apoptosis is induced (see, e.g., Non-Patent Document 3).

Moreover, it has also been found that anti-cancer agents that act specifically on the G-tail cause shortening of the G-tail without telomere shortening, leading to cancer cell death (see, e.g., Non-Patent Document 4). It is considered from these results that a drug or a stress that causes DNA damage transmits a signal to cells via the G tail and causes various cellular responses. In addition, it is also known that the tumor suppressor gene product p53 whose mutation is known in many cancers is bound to the G tail (see, e.g., Non-Patent Document 5), and it is clear that the change of the G tail functions as a signal also in cancers and diseases associated with aging.

### CITATION LIST

### Patent Literature

[Patent Literature 1] Japanese Laid-Open Patent Publication No. 5652850

### Non-Patent Literature

[Non-Patent Literature 1] Griffith J D, Comeau L, Rosenfield S, Stansel R M, Bianchi A, Moss H and de Lange T., Cell: 97(1999), 503-14.
[Non-Patent Literature 2] van Steensel B, Smogorzewska A and de Lange T., 92(1998), Cell: 401-13.
[Non-Patent Literature 3] Karlseder J, Broccoli D, Dai Y, Hardy S and de Lange T., Science: 283(1999), 1321-5.
[Non-Patent Literature 4] Gomez D, Paterski R, Lemarteleur T, Shin-Ya K, Mergny J L and Riou J F., J Biol Chem: 279(2004), 41487-94.
[Non-Patent Literature 5] Stansel R M, Subramanian D and Griffith J D., J Biol Chem: 277(2002), 11625-8.

### SUMMARY OF INVENTION

### Technical Problem

As described above, since a telomere-binding protein is important in maintaining the G tail, it is considered that inhibition of the telomere-binding protein may possibly be utilized for diagnosis of various diseases and development of therapeutic agents. However, a compound that inhibits the telomere-binding protein is not known.

The present invention has been made in view of the above-described problems, and its object is to obtain a compound that inhibits a telomere-binding protein, and further, to allow the compound to be applied to the diagnosis and treatment of diseases.

### Solution to Problem

In order to achieve the above-described object, the prevent inventors have intensively studied and resultantly found a compound that inhibits a telomere-binding protein, completing the present invention.

Specifically, the compound according to the present invention is characterized by being a compound represented by the following chemical formula.

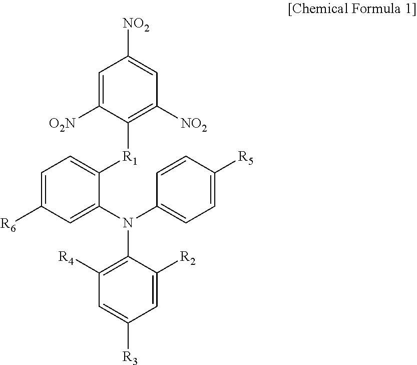

In the above-described chemical formula,

R₁ is oxygen or sulfur, and

R₂ to R₆ are each independently selected from hydrogen, an alkyl group having 1 to 6 carbon atoms, an alkoxy group having 1 to 6 carbon atoms, an acyl group having 1 to 6 carbon atoms and a nitro group.

Further, it is preferable for the compound according to the present invention that, in the above-described chemical formula,

R₁ is oxygen or sulfur,

R₂ and R₄ are each independently hydrogen or a nitro group,

R₃ is hydrogen, a nitro group, a methyl group, a methoxy group or a butyl group,

R₅ is hydrogen, a methyl group, a methoxy group or an acetyl group, and

R₆ is hydrogen or a butyl group.

Further, the compound according to the present invention is more preferably represented by any one of the following chemical formulae.

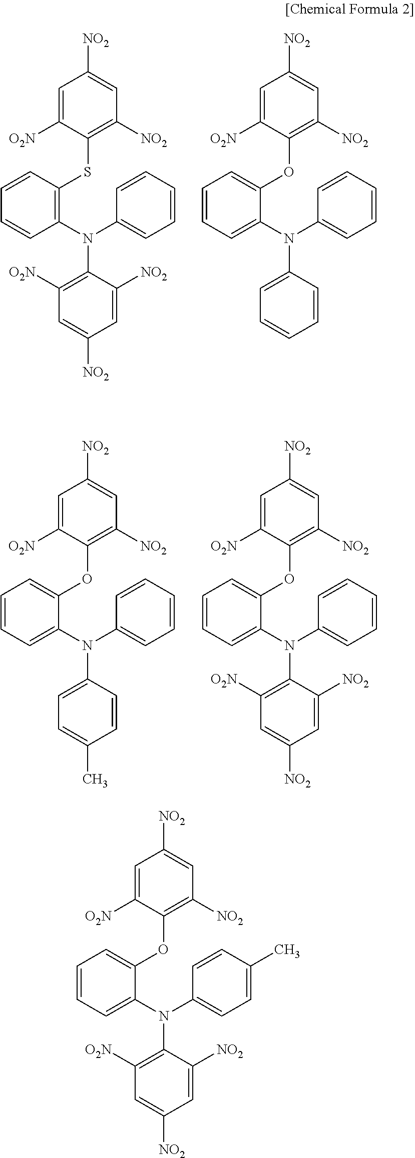

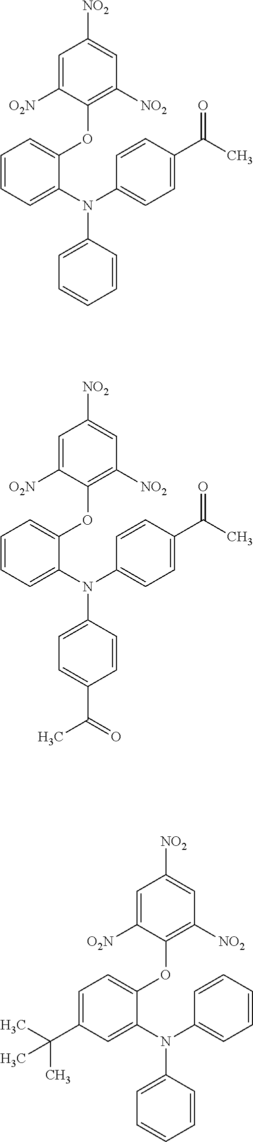

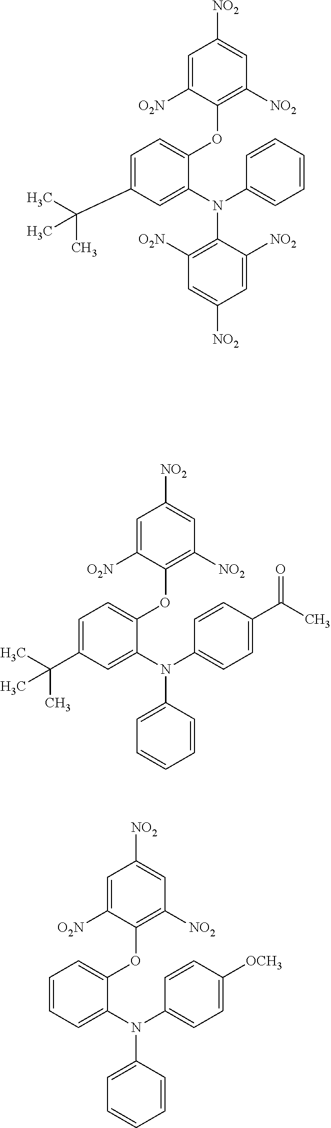 [SMILES: CC(C)(C)c1ccc(Oc2c([N+](=O)[O-])cc([N+](=O)[O-])cc2[N+](=O)[O-])c(N(c2ccccc2)c2c([N+](=O)[O-])cc([N+](=O)[O-])cc2[N+](=O)[O-])c1 ; CC(=O)c1ccc(N(c2ccccc2)c2cc(C(C)(C)C)ccc2Oc2c([N+](=O)[O-])cc([N+](=O)[O-])cc2[N+](=O)[O-])cc1 ; COc1ccc(N(c2ccccc2)c2ccccc2Oc2c([N+](=O)[O-])cc([N+](=O)[O-])cc2[N+](=O)[O-])cc1]

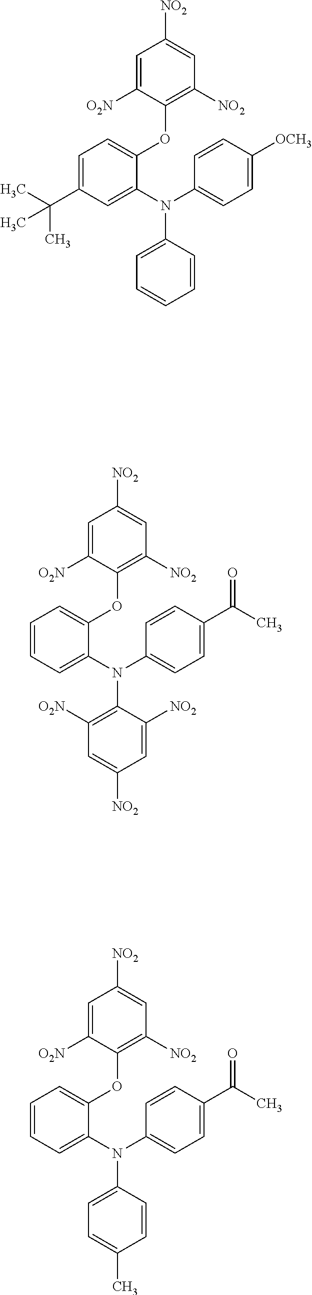 [SMILES: COc1ccc(N(c2ccccc2)c2cc(C(C)(C)C)ccc2Oc2c([N+](=O)[O-])cc([N+](=O)[O-])cc2[N+](=O)[O-])cc1 ; CC(=O)c1ccc(N(c2ccccc2Oc2c([N+](=O)[O-])cc([N+](=O)[O-])cc2[N+](=O)[O-])c2c([N+](=O)[O-])cc([N+](=O)[O-])cc2[N+](=O)[O-])cc1 ; CC(=O)c1ccc(N(c2ccc(C)cc2)c2ccccc2Oc2c([N+](=O)[O-])cc([N+](=O)[O-])cc2[N+](=O)[O-])cc1]

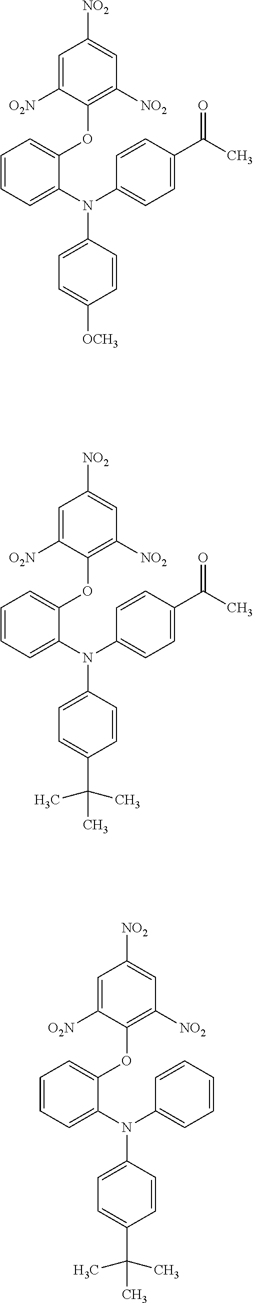 [SMILES: COc1ccc(N(c2ccc(C(C)=O)cc2)c2ccccc2Oc2c([N+](=O)[O-])cc([N+](=O)[O-])cc2[N+](=O)[O-])cc1 ; CC(=O)c1ccc(N(c2ccc(C(C)(C)C)cc2)c2ccccc2Oc2c([N+](=O)[O-])cc([N+](=O)[O-])cc2[N+](=O)[O-])cc1 ; CC(C)(C)c1ccc(N(c2ccccc2)c2ccccc2Oc2c([N+](=O)[O-])cc([N+](=O)[O-])cc2[N+](=O)[O-])cc1]

With the compound according to the present invention, the loop formation in the G tail can be inhibited since the compound can inhibit a telomere-binding protein from binding to telomere DNA. As a result, shortening of the G tail can be promoted, and cell aging and cell death can be induced. Hence, the compound according to the present invention may possibly be used as a reagent for inducing cell aging or cell death, and may possibly be applied to the development of therapeutic agents for various diseases such as cancers.

he telomere-binding protein inhibitor according to the present invention is characterized by containing any of the above-described compounds.

In the telomere-binding protein inhibitor according to the present invention, the telomere-binding protein is, for example, TRF1, TRF2 or POT1.

Since the telomere-binding protein inhibitor according to the present invention contains the above-described compound, it can inhibit the telomere-binding protein from binding to telomere DNA as described above. Hence, the loop formation in the G tail can be inhibited, the shortening of the G tail can be promoted, and cell aging and cell death can be induced.

From the above-described matters, it is considered that the compound according to the present invention can be used for treating or preventing cancers, and thus, the present invention relates to a pharmaceutical composition for treating or preventing cancers, which comprises the above-described compound, and also relates to the use of the above-described compound for production of a pharmaceutical composition for treating or preventing cancers. Furthermore, the present invention also relates to a method comprising administering the above-described compound to treat or prevent the cancer in a cancer patient.

### Advantageous Effects of Invention

The compound according to the present invention and the telomere-binding protein inhibitor containing the compound can inhibit the telomere-binding protein from binding to telomere DNA, and thus can inhibit the loop formation in the G tail. As a result, the shortening of the G tail can be promoted, and cell aging and cell death can be induced.

### BRIEF DESCRIPTION OF DRAWINGS

FIG. 1 is a view for explaining a DSE-FRET assay in an example of the present invention.

FIG. 2 is a view for explaining a DSE-FRET assay in an example of the present invention.

FIG. 3 is a view showing the result of a chromatin immunoprecipitation (ChIP) assay for examining the telomere binding inhibitory effect of TRF2 by the compound #198 which is a compound according to the present invention. FIG. 3(a) is a photograph of a membrane showing the result of the ChIP assay, and FIG. 3(b) is a graph showing the result obtained by quantifying the signal using an image analysis software on the result shown in (a) and calculating the telomeric DNA amount in the immunoprecipitate with the TRF2 antibody with respect to 10% input.

FIG. 4 is a view showing the result of a fluorescent immunostaining test for examining the telomere binding inhibitory effect of TRF2 by the compound #198 which is a compound according to the present invention. FIG. 4(a) is a photograph obtained with a fluorescence microscope, and FIG. 4(b) is a graph showing the result obtained by measuring the number of TRF2 foci in the nucleus using an image analysis software on the result shown in (a).

FIG. 5 is a view showing the result of a telomere FISH test for examining the telomere binding inhibitory effect of TRF2 by the compound #198 which is a compound according to the present invention. FIG. 5(a) is a photograph obtained with a fluorescence microscope, and FIG. 5(b) is a graph showing the result obtained by measuring 53BP1(TIF) localized in telomere using an image analysis software on the result shown in (a).

FIG. 6 is a view showing the result of a FACS test for examining the effect of inducing cell apoptosis by the compound #198 which is a compound according to the present invention.

FIG. 7 is a view showing the result of a Western blot test for examining the effect of inducing cell apoptosis by the compound #198 which is a compound according to the present invention.

FIG. 8 is a view showing the result of a cell growth test for examining the cell growth inhibitory effect by the compound #198 which is a compound according to the present invention.

FIG. 9 is a view showing the result of a colony formation assay for examining the cell growth inhibitory effect by the compound #198 which is a compound according to the present invention. FIG. 9(a) is a photograph showing the culture plate under each condition, and FIG. 9(b) is a graph showing the result obtained by measuring the number of colonies from the result shown in (a).

### DESCRIPTION OF EMBODIMENTS

Hereinafter, embodiments for carrying out the present invention will be described with reference to the drawings. The description of the preferred embodiments below is substantially only exemplary and is not intended to limit the present invention, its application method or its use.

The compound according to this embodiment is a compound that inhibits a telomere-binding protein. Further, the compound according to this embodiment is represented by the following chemical formula.

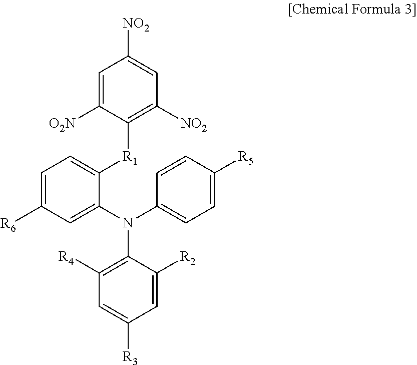

In the above-described chemical formula,

R₁ is oxygen or sulfur, and

R₂ to R₆ are each independently selected from hydrogen, an alkyl group having 1 to 6 carbon atoms, an alkoxy group having 1 to 6 carbon atoms, an acyl group having 1 to 6 carbon atoms and a nitro group.

Further, it is preferable for the compound according to the present embodiment that, in the above-described chemical formula,

R₁ is oxygen or sulfur,

R₂ and R₄ are each independently hydrogen or a nitro group,

R₃ is hydrogen, a nitro group, a methyl group, a methoxy group or a butyl group,

R₅ is hydrogen, a methyl group, a methoxy group or an acetyl group, and

R₆ is hydrogen or a butyl group.

In the present embodiment, the telomere-binding protein is, for example, TRF1, TRF2, POT1 or the like. In addition, in the present embodiment, the above-described compound particularly inhibits the telomere-binding protein from binding to telomere.

Since the compound of the present embodiment has such characteristics, it can be used for a telomere-binding protein inhibitor, and further, can be used in a pharmaceutical composition for treating or preventing cancers.

### EXAMPLES

Examples will be shown below for illustrating the compound and the telomere-binding protein inhibitor containing the same according to the present invention in detail.

First, compounds that inhibit a telomere-binding protein were screened using a DSE-FRET assay (see the above-described Patent Document 1, especially the first embodiment). As the candidate compounds, 12212 compounds in the compound library held by National Institute of Advanced Industrial Science and Technology were used. The principle of the DSE-FRET assay is described in Patent Document 1, and it will be briefly described also below.

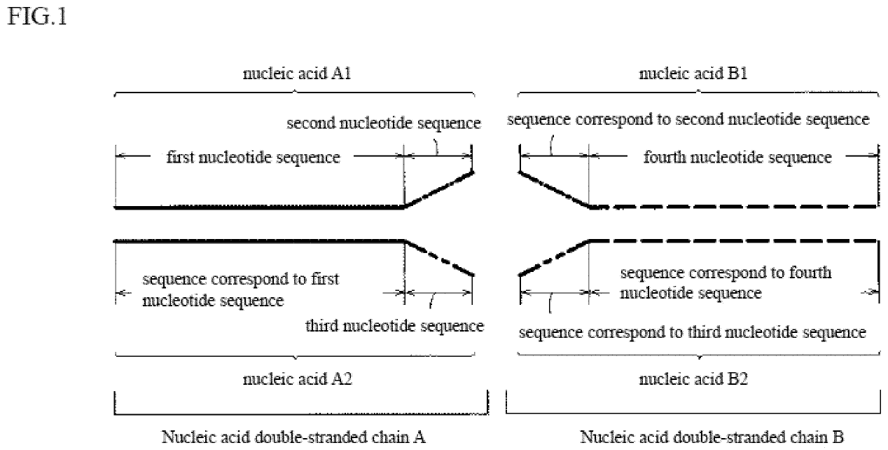

As shown in Patent Document 1, the DSE-FRET assay is a method characterized by measuring the amount of a new nucleic acid double-stranded chain generated by the structural change of a complex (nucleic acid double-stranded complex) in which two nucleic acid double-stranded portions (nucleic acid double-stranded chain A and nucleic acid double-stranded chain B) are bound to each other at their terminal sequences. As shown in FIG. 1 of the present application, the nucleic acid double-stranded chain A is a nucleic acid double-stranded chain which is composed of a nucleic acid A1 as a nucleic acid single-stranded chain and a nucleic acid A2 as a nucleic acid single-stranded chain, and capable of binding to the nucleic acid double-stranded chain B at the terminal sequence. The nucleic acid double-stranded chain B is a nucleic acid double-stranded chain which is composed of a nucleic acid B1 as a nucleic acid single-stranded chain and a nucleic acid B2 as a nucleic acid single-stranded chain, and capable of binding to the nucleic acid double-stranded chain A at the terminal sequence.

Each of the nucleic acid A1, the nucleic acid A2, the nucleic acid B1 and the nucleic acid B2 can be designed as follows.

Nucleic acid A1: a nucleic acid single-stranded chain having a first nucleotide sequence and a second nucleotide sequence (terminal sequence).

Nucleic acid A2: a nucleic acid single-stranded chain having a sequence corresponding to the first nucleotide sequence, and a third nucleotide sequence (terminal sequence).

Nucleic acid B1: a nucleic acid single-stranded chain having a sequence (terminal sequence) corresponding to the second nucleotide sequence, and a fourth nucleotide sequence.

Nucleic acid B2: a nucleic acid single-stranded chain having a sequence (terminal sequence) corresponding to the third nucleotide sequence, and a sequence corresponding to the fourth nucleotide sequence.

Embodiments of the specific structures of the nucleic acid A1, the nucleic acid A2, the nucleic acid B1 and the nucleic acid B2 are as shown in FIG. 1 of the present application.

The nucleic acid A1, the nucleic acid A2, the nucleic acid B1, and the nucleic acid B2 are designed to have a binding site for a nucleic acid-binding protein. In this example, for the purpose of evaluating the binding of the telomere-binding protein to the telomere sequence and its inhibition, the binding site of the nucleic acid-binding protein was the binding sequence of the telomere-binding protein, particularly TRF2. Details of the sequence will be described later.

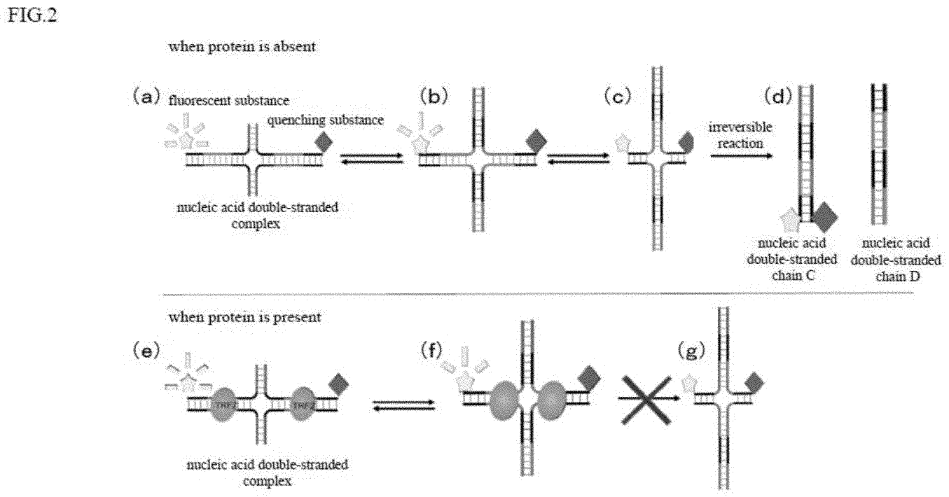

The method utilizes a fact that the structural change between the nucleic acid double-stranded chains is inhibited by the binding of the nucleic acid-binding protein. A nucleic acid double-stranded complex in which a nucleic acid double-stranded chain A constituted of a nucleic acid A1 and a nucleic acid A2 and a nucleic acid double-stranded chain B constituted of a nucleic acid B1 and a nucleic acid B2 are bound to each other at their terminal sequences shows a structural change by a chain exchange reaction. Specifically, a nucleic acid double-stranded complex in which the terminal sequences of a nucleic acid double-stranded chain A and a nucleic acid double-stranded chain B are bound to each other shown in FIG. 2(a) gets a structure shown in FIG. 2(b) changed by a chain exchange reaction, and further gets a structure shown in FIG. 2(c) changed by the chain exchange reaction. The structural change of FIGS. 2(a) to 2(c) is a reversible change. From the structure shown in FIG. 2(c), a nucleic acid double-stranded chain C constituted of a nucleic acid A1 and a nucleic acid B1 and a nucleic acid double-stranded chain D constituted of a nucleic acid A2 and a nucleic acid B2 are generated, as the chain exchange reaction further proceeds, as shown in FIG. 2(d). The structural change from FIGS. 2(c) to 2(d) is an irreversible reaction. In contrast, when a nucleic acid-binding protein (telomere-binding protein) binds to either a nucleic acid double-stranded chain A or a nucleic acid double-stranded chain B, the above-described structural change is inhibited. Specifically, when a nucleic acid-binding protein is bound to a nucleic acid double-stranded complex constituted of a nucleic acid double-stranded chain A and a nucleic acid double-stranded chain B as shown in FIG. 2(e), the structural change proceeds to some extent by a chain exchange reaction as shown in the FIG. 2(f), however, the protein inhibits the chain exchange reaction at the portion where the protein is bound. Therefore, the chain exchange reaction is interrupted at this portion, and as a result, the structure cannot be changed into the structure shown in FIG. 2(g).

Therefore, by measuring the amounts of nucleic acid double-stranded chains A and B and nucleic acid double-stranded chains C and D, the degree of structural change due to the above-described chain exchange reaction, that is, the degree of binding of a telomere-binding protein to telomere sequence can be measured. Such measurement can be easily performed by labeling the nucleic acid double-stranded chain. It becomes possible to measure the degree of binding of a telomere-binding protein to telomere sequence, for example, by labeling the 5′ end of a nucleic acid A1 with a fluorescent substance and labeling the 3′ end of a nucleic acid B1 with a quenching substance, and measuring the fluorescence intensity of the fluorescent substance, though the measurement method is not limited to this.

For example, in the case wherein the 5′ end of a nucleic acid A1 is labeled with a fluorescent substance and the 3′ end of a nucleic acid B1 is labeled with a substance which quenches the above-described fluorescent substance, fluorescence is emitted when a nucleic acid double-stranded complex is formed of nucleic acid double-stranded chains A and B as shown in FIG. 2(a). On the other hand, when the chain exchange reaction proceeds, and, when the 5′ end of a nucleic acid A1 comes closer to the 3′ end of a nucleic acid B1, that is, when the fluorescent substance comes closer to the quenching substance as shown in FIGS. 2(b) and 2(c), the fluorescence intensity is more reduced. Furthermore, when the chain exchange reaction proceeds, and, when a nucleic acid double-stranded chain C and a nucleic acid double-stranded chain D as final products are formed as shown in FIG. 2(d), quenching is caused by labeling of a nucleic acid B1. Hence, by measuring the fluorescence value, the amounts of a nucleic acid double-stranded complex, a nucleic acid double-stranded chain C and a nucleic acid double-stranded chain D can be easily measured. The combination of the positions labeled with a fluorescent substance and a quenching substance is not limited to the combination of the positions described above, and may be any position where the fluorescent substance and the quenching substance are close to or apart from each other as the chain exchange reaction proceeds. Further, the quenching substance and the fluorescent substance can be exchanged.

As described above, when TRF2 does not bind to any of the nucleic acids having a TRF2 binding site (when binding is inhibited), the above-described chain exchange reaction proceeds and the positions of a fluorescent substance and a quenching substance come close to each other, to cause quenching, while when TRF2 binds, the above-described chain exchange does not proceed, and a fluorescent substance and a quenching substance are apart from each other, thus, fluorescence is emitted. That is, according to the present method, it is possible to measure the degree of binding of TRF2 or its inhibition based on the fluorescence intensity.

The method and the result of the DSE-FRET assay conducted in the present example are described below.

First, a synthetic oligonucleotide TLM-06 corresponding to the nucleic acid A2 and a synthetic oligonucleotide TLM-01-5F corresponding to the nucleic acid A1 of which 5′ end is labeled with FAM (fluorescent substance) were mixed in 20 μL of a double-stranded chain forming solution (10 mM HEPES-NaOH (pH 7.9), 50 mM KCl, 30 mM NaCl, 0.1 mM EDTA, 2.5 mM DTT, 10% glycerol, 0.05% IGEPAL CA-630). Thereafter, a double-stranded chain TO1F/06 corresponding to the above-described nucleic acid double-stranded chain A having a single-stranded chain at the end was prepared by heat denaturation and annealing. TO1C/06 has a TRF2 binding sequence. Further, a synthetic oligonucleotide TLM-05 corresponding to the nucleic acid B2 and a synthetic oligonucleotide TLM-02-3D corresponds to the nucleic acid B1 of which 3′ end is labeled with Dabcyl (quenching substance) were mixed in 20 μL of a double-stranded chain forming solution (10 mM HEPES-NaOH (pH 7.9), 50 mM KCl, 30 mM NaCl, 0.1 mM EDTA, 2.5 mM DTT, 10% glycerol, 0.05% IGEPAL CA-630). Thereafter, a double-stranded chain TO2D/05 corresponding to the above-described nucleic acid double-stranded chain B having a single-stranded chain at the end was prepared by heat denaturation and annealing. TO2D/05 has a TRF2 binding sequence. The synthetic oligonucleotides were all used in an amount of 20 pmol. Hereinafter, for all the labels, those produced by requesting synthesis from Japan Bio Services Co., Ltd. were used.

The heat denaturation and annealing were performed under the following temperature conditions.

95° C., 120 seconds; 90° C., 30 seconds; 85° C., 90 seconds; 80° C., 90 seconds; 77° C., 90 seconds; 75° C., 90 seconds; 72° C., 90 seconds; 70° C., 90 seconds; 67° C., 90 seconds; 65° C., 90 seconds; 62° C., 90 seconds; 60° C., 90 seconds; 57° C., 90 seconds; 55° C., 90 seconds; 52° C., 90 seconds; 50° C., 90 seconds; 47° C., 90 seconds; 45° C., 90 seconds; 42° C., 90 seconds; 40° C., 90 seconds; 37° C., 90 seconds; 35° C., 90 seconds; 32° C., 90 seconds; 30° C., 90 seconds.

The sequences used are as follows. The underlined portion is a TRF2 binding sequence, and the lower-case portion is a sequence forming a single-stranded chain when a nucleic acid complex is formed.

TLM-01-5F:
(SEQ ID NO: 1)
5′ FAM-AGTTGAGTTA GGGTTAGGGT TAGGGTTAGG GCAGGcggtg
tctcgctcgc 3′
TLM-02-3D:
(SEQ ID NO: 2)
5′ gcgagcgaga caccgCCTGC CCTAACCCTA ACCCTAACCC
TAACTCAACT-Dabcyl 3′
TLM-05:
(SEQ ID NO: 3)
5′ AGTTGAGTTA GGGTTAGGGT TAGGGTTAGG GCAGGcacca
caccattccc 3′
TLM-06:
(SEQ ID NO: 4)
5′ gggaatggtg tggtgCCTGC CCTAACCCTA ACCCTAACCC
TAACTCAACT 3′

100 fmol of T01F/06 and 50 μM of a candidate compound were mixed in a reaction solution (10 mM HEPES-NaOH pH 7.9, 150 mM KCl, 0.1 mM EDTA, 5 mM DTT, 10% glycerol, 0.05% IGEPAL CA-630, 20 μL) and reacted at 25° C. for 30 minutes. Thereafter, to T01F/06 was added 100 fmol of TO2D/05 to make 50 μL, then, the mixture was reacted at 25° C. for 120 minutes. For measurement of the fluorescence value of Cy3, a fluorescence plate reader EnVision (manufactured by Perkin Elmer) was used.

As a result of screening candidate compounds, a compound #10 having the following chemical formula was obtained as a compound which shows high fluorescence intensity detected, that is, which inhibits the binding of TRF2 to its binding site.

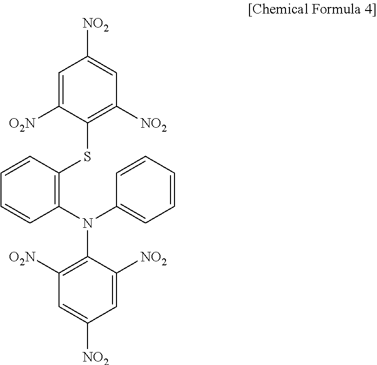 [SMILES: O=[N+]([O-])c1cc([N+](=O)[O-])c(Sc2ccccc2N(c2ccccc2)c2c([N+](=O)[O-])cc([N+](=O)[O-])cc2[N+](=O)[O-])c([N+](=O)[O-])c1]

In addition, based on the structure of the compound #10 obtained, compounds having a structure similar to this were synthesized, and subjected to the above-described screening. As a result, compounds which show high fluorescence intensity detected and which inhibit the binding of TRF2 to its binding site are as shown in Table 1 below. The reduction rate of the fluorescence intensity when each compound was mixed with the reaction solution was calculated as the inhibition rate, with reference to the fluorescence intensity of the control in which the candidate compound was not mixed with the reaction solution.

TABLE 1

| | | | inhibition |
|---|---|---|---|
| | | | rate (%) |
| number of | | | in DSE |
| compound | | molecular | FRET |
| (50 μM) | structure | weight | assay |
| #10 | 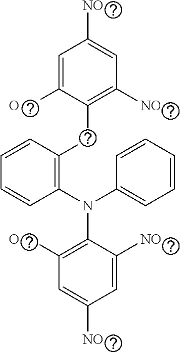 [SMILES: O=[N+]([O-])c1cc([N+](=O)[O-])c(Cc2ccccc2N(c2ccccc2)c2c([N+](=O)[O-])cc([N+](=O)[O-])cc2[N+](=O)[O-])c([N+](=O)[O-])c1] | 623.47 | 53.2 |
| #144 | 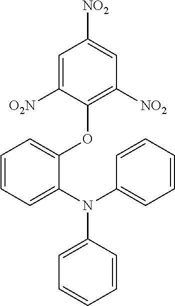 [SMILES: O=[N+]([O-])c1cc([N+](=O)[O-])c(Oc2ccccc2N(c2ccccc2)c2ccccc2)c([N+](=O)[O-])c1] | 472.41 | 38.3 |
| #145 | 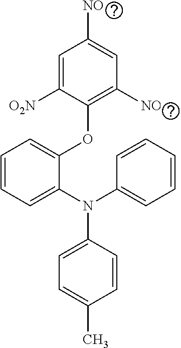 [SMILES: Cc1ccc(N(c2ccccc2)c2ccccc2Oc2c([N+](=O)[O-])cc([N+](=O)[O-])cc2[N+](=O)[O-])cc1] | 486.44 | 38.7 |
| #151 | 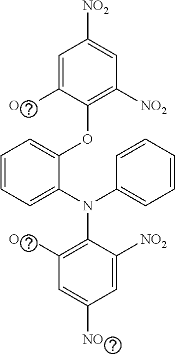 [SMILES: O=[N+]([O-])c1cc([N+](=O)[O-])c(Oc2ccccc2N(c2ccccc2)c2c([N+](=O)[O-])cc([N+](=O)[O-])cc2[N+](=O)[O-])c([N+](=O)[O-])c1] | 607.4 | 49.6 |
| #153 | 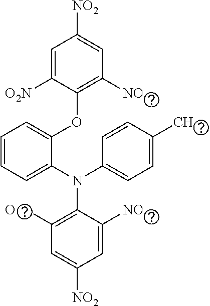 [SMILES: Cc1ccc(N(c2ccccc2Oc2c([N+](=O)[O-])cc([N+](=O)[O-])cc2[N+](=O)[O-])c2c([N+](=O)[O-])cc([N+](=O)[O-])cc2[N+](=O)[O-])cc1] | 621.43 | 22.6 |
| #168 | 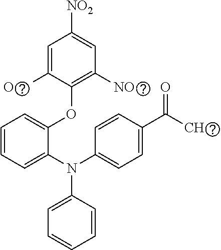 [SMILES: CC(=O)c1ccc(N(c2ccccc2)c2ccccc2Oc2c([N+](=O)[O-])cc([N+](=O)[O-])cc2[N+](=O)[O-])cc1] | 514.44 | 25.5 |
| #171 | 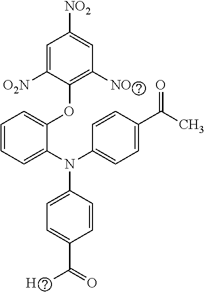 [SMILES: CC(=O)c1ccc(N(c2ccc(C(C)=O)cc2)c2ccccc2Oc2c([N+](=O)[O-])cc([N+](=O)[O-])cc2[N+](=O)[O-])cc1] | 556.48 | 24.7 |
| #192 | 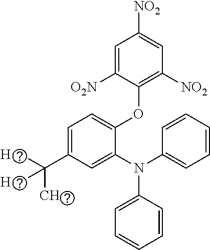 [SMILES: CC(C)(C)c1ccc(Oc2c([N+](=O)[O-])cc([N+](=O)[O-])cc2[N+](=O)[O-])c(N(c2ccccc2)c2ccccc2)c1] | 528.51 | 33.6 |
| #198 | 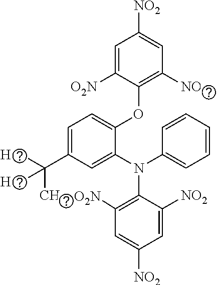 [SMILES: CC(C)(C)c1ccc(Oc2c([N+](=O)[O-])cc([N+](=O)[O-])cc2[N+](=O)[O-])c(N(c2ccccc2)c2c([N+](=O)[O-])cc([N+](=O)[O-])cc2[N+](=O)[O-])c1] | 663.51 | 62.2 |
| #201 | 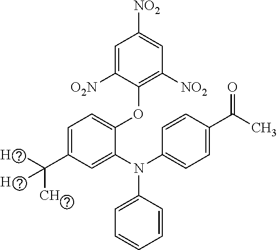 [SMILES: CC(=O)c1ccc(N(c2ccccc2)c2cc(C(C)(C)C)ccc2Oc2c([N+](=O)[O-])cc([N+](=O)[O-])cc2[N+](=O)[O-])cc1] | 570.56 | 23.0 |
| #204 | 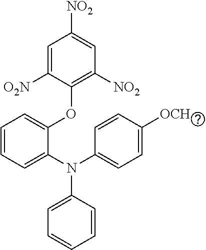 [SMILES: COc1ccc(N(c2ccccc2)c2ccccc2Oc2c([N+](=O)[O-])cc([N+](=O)[O-])cc2[N+](=O)[O-])cc1] | 502.44 | 20.4 |
| #207 | 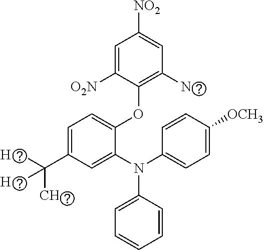 | 558.55 | 23.7 |
| #224 | 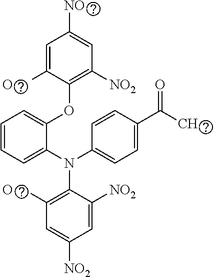 [SMILES: CC(=O)c1ccc(N(c2ccccc2Oc2c([N+](=O)[O-])cc([N+](=O)[O-])cc2[N+](=O)[O-])c2c([N+](=O)[O-])cc([N+](=O)[O-])cc2[N+](=O)[O-])cc1] | 635.43 | 71.7 |
| #225 | 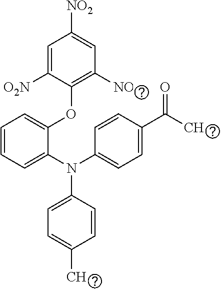 [SMILES: CC(=O)c1ccc(N(c2ccc(C)cc2)c2ccccc2Oc2c([N+](=O)[O-])cc([N+](=O)[O-])cc2[N+](=O)[O-])cc1] | 514.46 | 36.9 |
| #226 | 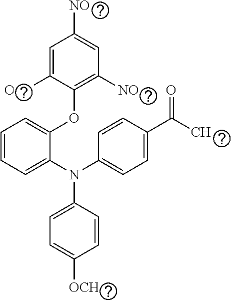 | 530.46 | 19.3 |
| #227 | 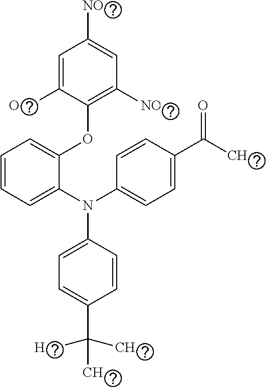 [SMILES: CC(=O)c1ccc(N(c2ccc(C(C)(C)C)cc2)c2ccccc2Oc2c([N+](=O)[O-])cc([N+](=O)[O-])cc2[N+](=O)[O-])cc1] | 556.64 | 18.4 |
| #228 | 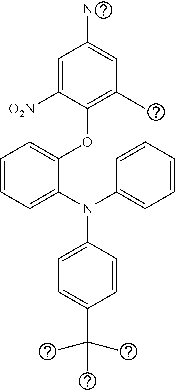 [SMILES: Cc1cc([N+](=O)[O-])cc([N+](=O)[O-])c1Oc1ccccc1N(c1ccccc1)c1ccc(C(C)(C)C)cc1] | 514.51 | 11.8 |

It can be seen that the compounds shown in Table 1 above have the following chemical formula as a common skeleton.

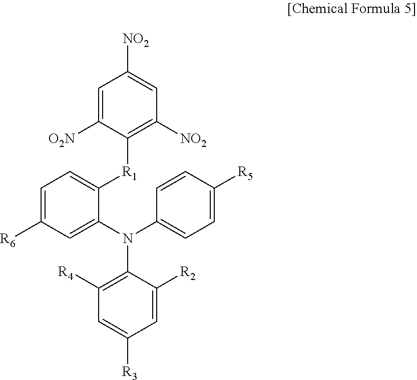

In the above-described chemical formula,

R₁ is oxygen or sulfur,

R₂ and R₄ are each independently hydrogen or a nitro group,

R₃ is hydrogen, a nitro group, a methyl group, a methoxy group or a butyl group,

R₅ is hydrogen, a methyl group, a methoxy group or an acetyl group, and

R₆ is hydrogen or a butyl group.

Further, each of the above-described compounds is synthesized by a usual synthetic method. For example, the method for synthesizing a compound #198 is shown below.

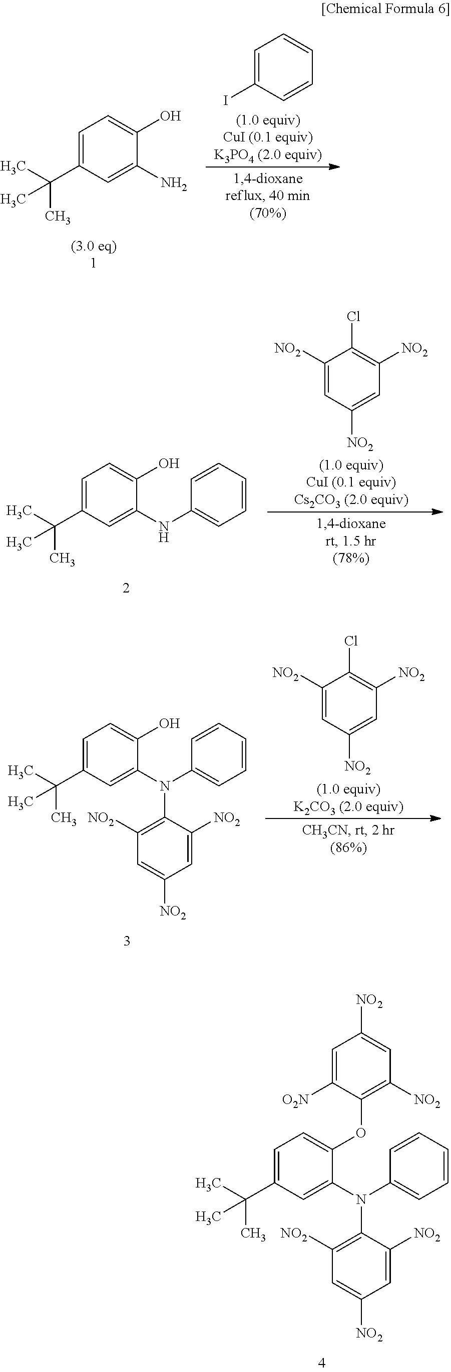

Other compounds can also be easily synthesized by those skilled in the art by partially modifying the above-described synthesis method for a compound #198, that is, by using those having a suitable substituent as aminophenol derivatives and halogenated aryls as raw materials. For example, in the case of a compound #207, a person skilled in the art can easily think of using 4-iodoanisole instead of 2,4,6-trinitrochlorobenzene in synthesis from 2 to 3 in [Chemical Formula 6] of the above-described synthesis method for a compound #198.

Here, specific synthesis methods for other compounds used in the present example are shown below.

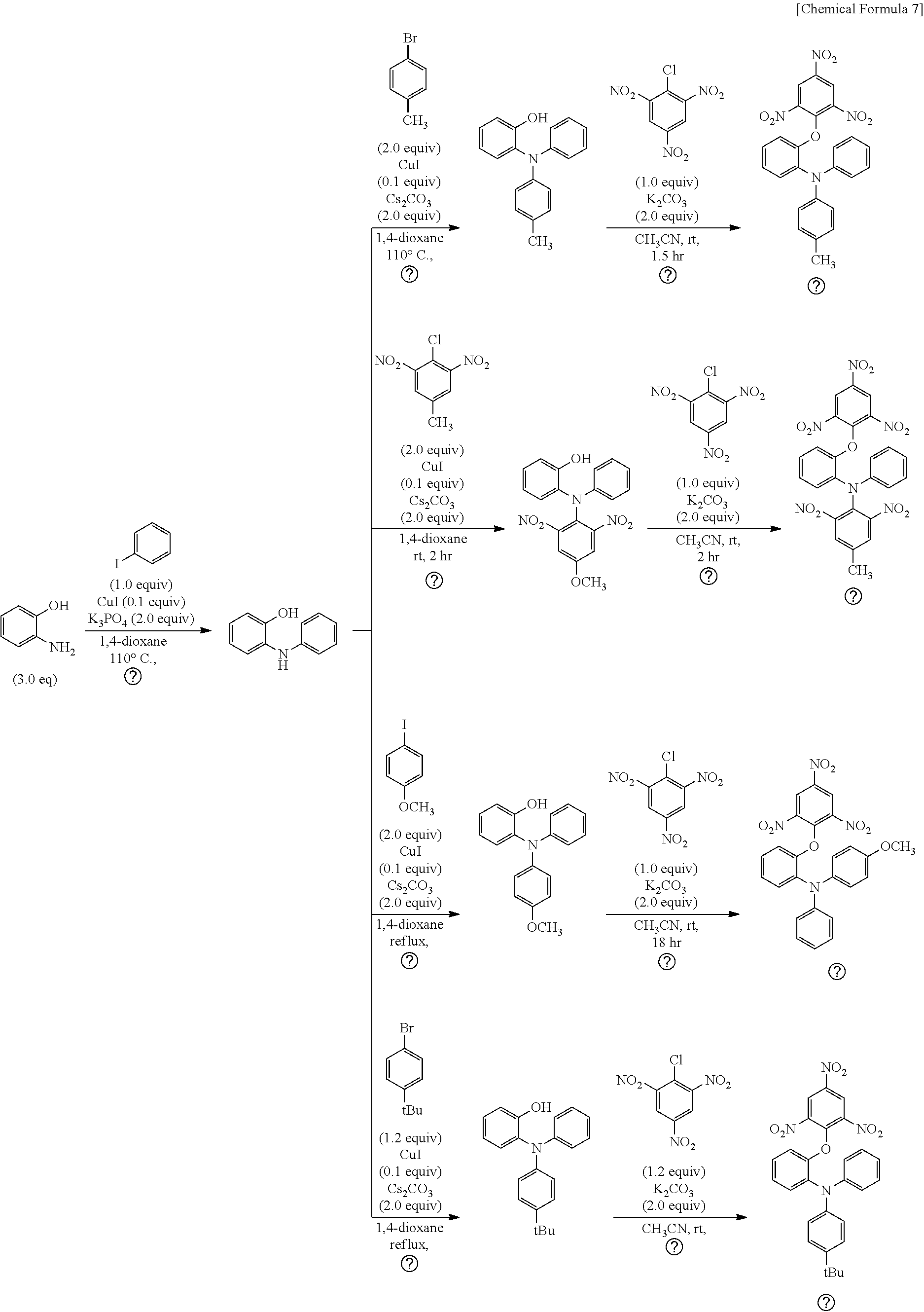

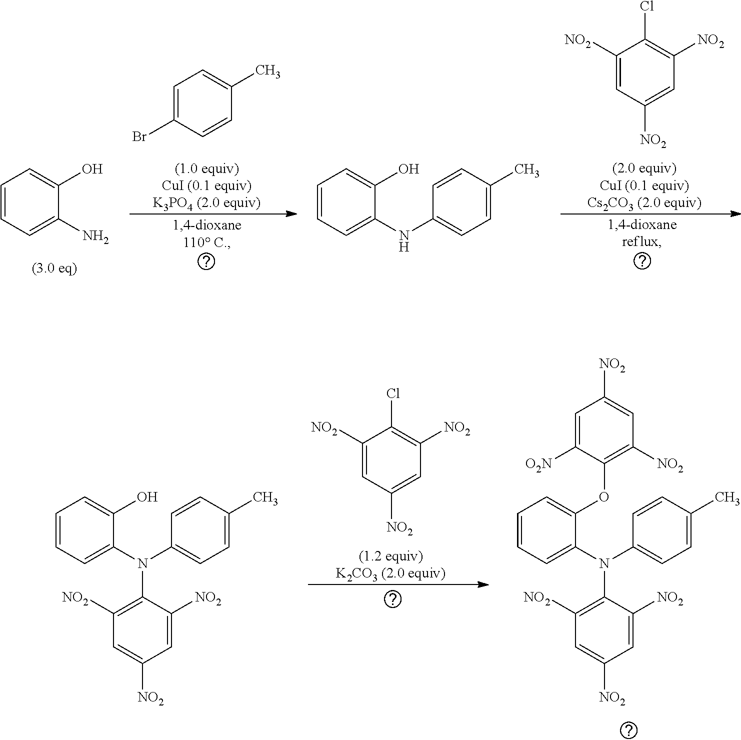

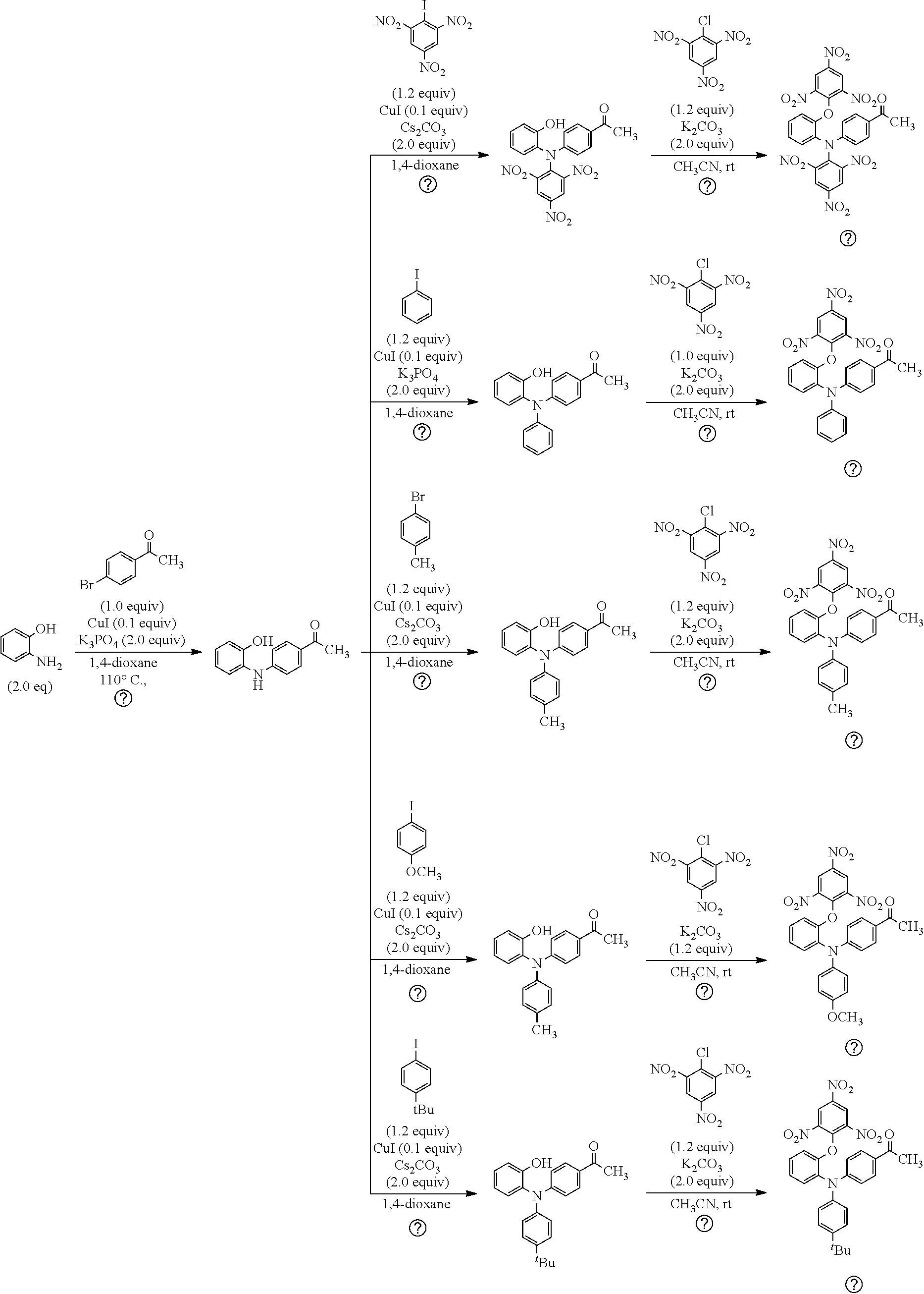

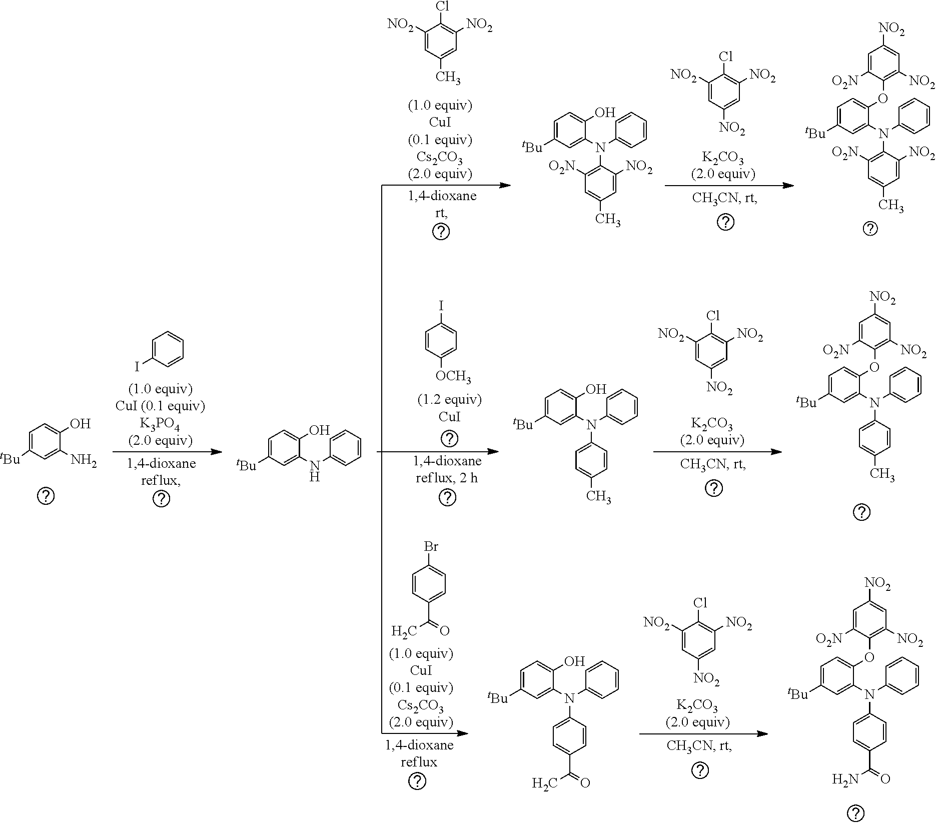

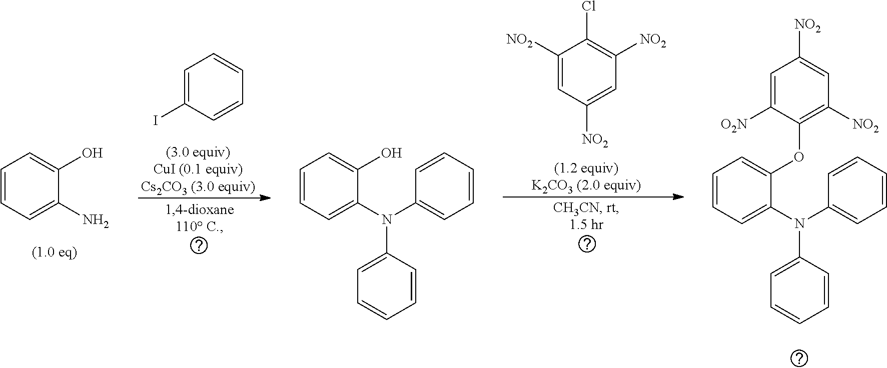 [SMILES: Nc1ccccc1O ; Ic1ccccc1 ; Oc1ccccc1N(c1ccccc1)c1ccccc1 ; O=[N+]([O-])c1cc([N+](=O)[O-])c(Cl)c([N+](=O)[O-])c1 ; O=[N+]([O-])c1cc([N+](=O)[O-])c(Oc2ccccc2N(c2ccccc2)c2ccccc2)c([N+](=O)[O-])c1]

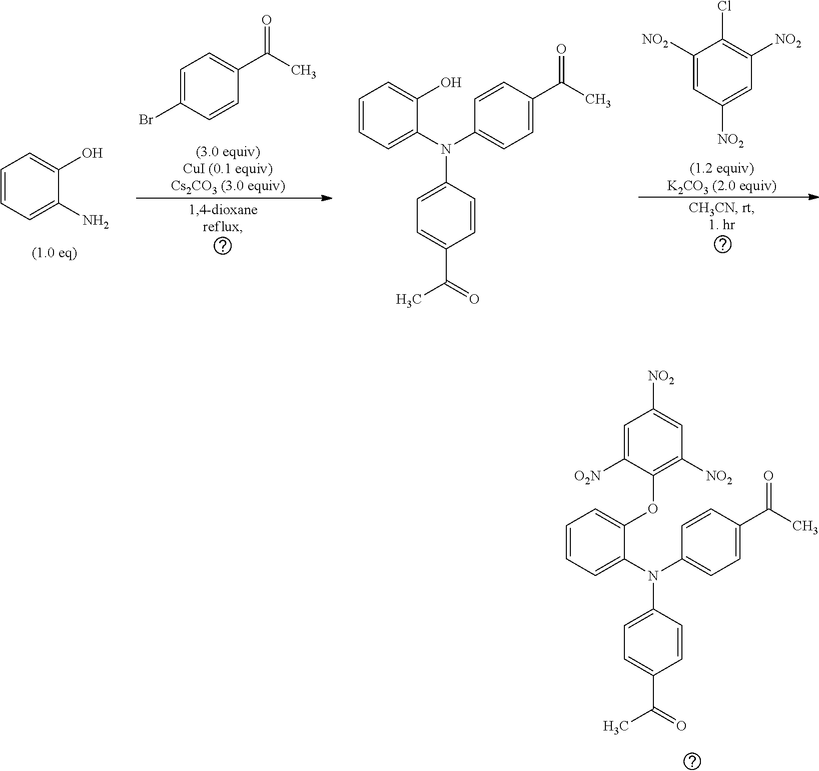

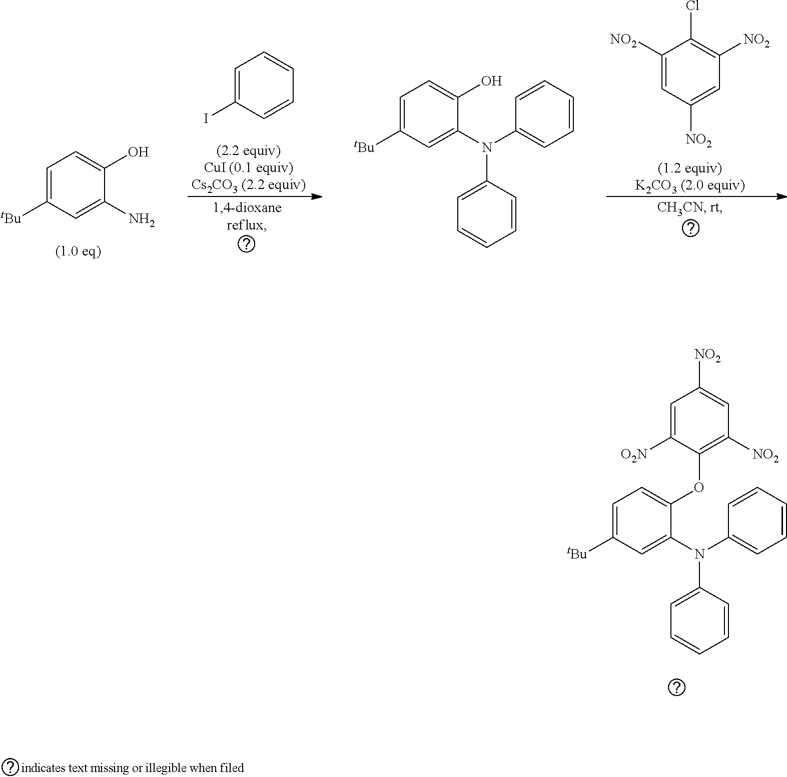 [SMILES: CC(C)(C)c1ccc(O)c(N)c1 ; Ic1ccccc1 ; CC(C)(C)c1ccc(O)c(N(c2ccccc2)c2ccccc2)c1 ; O=[N+]([O-])c1cc([N+](=O)[O-])c(Cl)c([N+](=O)[O-])c1 ; CC(C)(C)c1ccc(Oc2c([N+](=O)[O-])cc([N+](=O)[O-])cc2[N+](=O)[O-])c(N(c2ccccc2)c2ccccc2)c1]

Next, the TRF2 inhibitory effect was further examined using a compound #198 which was found to have a relatively high TRF2 inhibitory effect in the result of the above-described screening. For this examination, a chromatin immunoprecipitation test (ChIP assay) using a TRF2 antibody was conducted. The method and the result are described below.

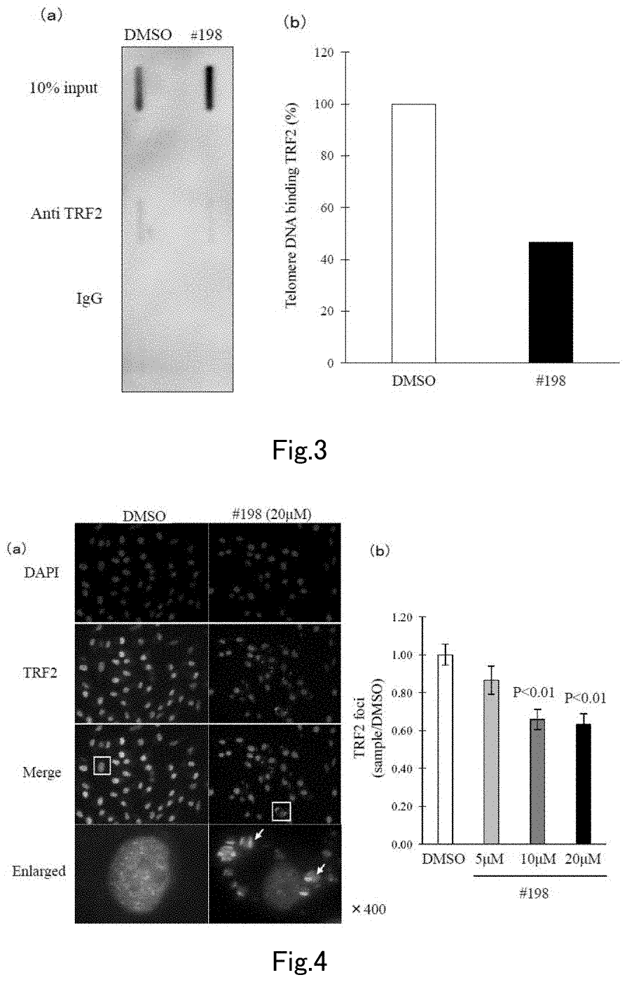

HeLa1.2.11 cells were treated with a compound #198 (20 μM) or DMSO as a control for 24 hours, then, fixed with 1% formaldehyde. Thereafter, the cells were dissolved with Lysis buffer (1% SDS, 10 mM EDTA (pH 8.0), 50 mM Tris-HCl (pH 8.0)), and chromatin was fragmented by ultrasonic wave. Thereafter, chromatin immunoprecipitation was performed using a TRF2 antibody (Santa Cruz Biotechnology, sc-8528) or normal mouse IgG (Santa Cruz Biotechnology, sc-2025), and DNA was adsorbed to Hybond N+membrane (Amersham Biosciences Corp., RPN 82B), then, it was reacted with a DIG-labeled telomere probe (obtained by labeling the 3′ end of 100 μmol of 3′-(CCCTAA)₄-5′ oligonucleotide with Digoxigenin using Dig oligo nucleotide Tailing kit (Roche, 03 353 583 910), and the signal was detected by Luminoanalyzer (ImageQuant, LAS 4000). The signal was quantified using an image analysis software ImageJ, and the amount of telomere DNA in the immunoprecipitate by the TRF2 antibody against 10% input was calculated. The result is shown in FIG. 3. FIG. 3(a) is a photograph of Hybond N+membrane, and FIG. 3(b) is a graph showing the result of calculating the telomeric DNA amount by quantifying the above-described signal.

As shown in FIG. 3, detection of telomere DNA was suppressed to 50% or less in the case where treated with a compound #198, compared with the control using DMSO. This result suggests that the compound #198 inhibits binding of TRF2 to telomeric DNA.

Next, intracellular localization of TRF2 with or without treatment with a compound #198 was examined by a fluorescent immunostaining method. The method and the result are described below.

HeLa1.2.11 cells seeded on a 8-well culture slide for tissue culture (Matsunami Glass Ind., Ltd., scs-008) were treated with a compound #198 (20 μM) or DMSO as a control for 24 hours. Thereafter, it was washed with PBS(−) twice, and treated with 0.25% TritonX-100 (WAKO, 591-12191)/PBS(−) on ice for 2 minutes, and treated with 4% paraformaldehyde (MERCK, 1.04005.1000)/PBS(−) at room temperature for 15 minutes, and then, washed with PBS(−) twice. Further, it was treated with 0.5% Triton X-100/PBS(−) on ice for 10 minutes, and then, washed with PBS(−) 5 times. Next, it was reacted with a TRF2 antibody (Novus, NB100-56506) diluted 200-fold with 3% BSA/0.05% Tween/PBS(−) at 37° C. for 1 hour, and then, washed with PBS(−) twice. Next, it was reacted with Alexa Fluor 488 Goat anti-mouse IgG (invitrogen, A11001) diluted 500-fold with 3% BSA/0.05% Tween/PBS(−) at room temperature for 45 minutes, and washed with PBS(−) three times, and reacted with 0.25 μg/mL DAPI (Dojindo Laboratories, 340-07971) at room temperature for 5 minutes, and washed with PBS(−) 3 times. After enclosing the slide glass, it was observed with a fluorescence microscope (Zwiss, Axiovert 200M). The number of TRF2 foci in the nucleus was measured using an image analysis software Columbus. The result is shown in FIG. 4. FIG. 4(a) is a photograph of cells obtained by using a fluorescence microscope, and in FIG. 4(a), an enlarged white square area is indicated by “Enlarged”, and an arrow in “Enlarged” indicates TRF2 existing outside the nucleus. FIG. 4(b) is a graph showing the ratio of the number of TRF2 foci when the ratio in the control is set to 1.00, obtained by measuring the number of TRF2 foci in the nucleus using a fluorescence microscope.

As shown in FIG. 4, in the DMSO-treated control, TRF2 is present in the nucleus stained with DAPI, then, it is believed that TRF2 is bound to the DNA in the nucleus. On the other hand, in cells treated with a compound #198, most of TRF2 is located outside the nucleus of the cell. This result suggests that the compound #198 inhibits TRF2 from binding to the TRF2 binding site of DNA in the nucleus.

Next, the influence of the compound #198 on telomere abnormality was examined using a telomere FISH method. Specifically, whether or not the compound #198 affects the localization of 53BP1 in telomere was examined using a telomere FISH method, because it is known that when abnormality occurs in telomere, TIF (telomere-induced DNA damage foci) characterized by localization of 53BP1 in telomere occurs. The method and the result are described below.

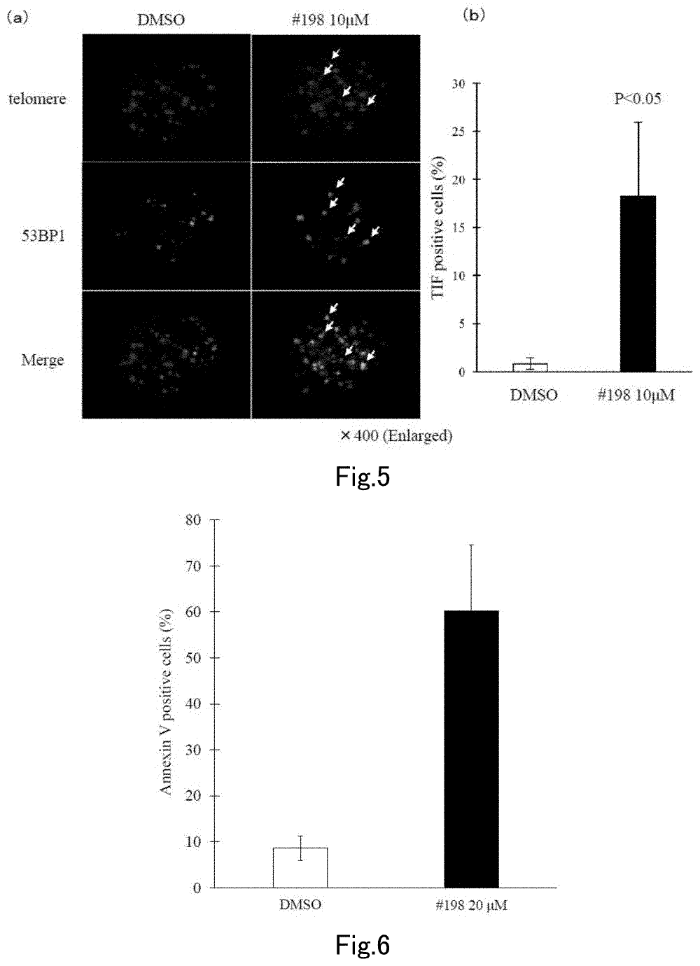

HeLa1.2.11 cells seeded on a 8-well culture slide for tissue culture were treated with a compound #198 (10 μM) or DMSO as a control for 24 hours. Thereafter, it was washed with PBS(−) twice, and treated with 0.25% TritonX-100 (WAKO, 591-12191)/PBS(−) on ice for 2 minutes, and treated with 4% paraformaldehyde (MERCK, 1.04005.1000)/PBS(−) at room temperature for 15 minutes. Thereafter, it was washed with PBS(−) twice, and was treated with 0.5% Triton X-100/PBS(−) on ice for 10 minutes, and then, washed with PBS(−) 5 times. Next, it was treated with Blocking solution (1 mg/ml BSA, 3% goat serum, 0.1% Triton X-100, 1 mM EDTA, pH 8.0) at room temperature for 30 minutes, and reacted with 53BP1 antibody (Novus, NB100-304) diluted 1000-fold with Blocking solution at 37° C. for 1 hour, and then, washed with PBS(−) twice. Next, it was reacted with Alexa Fluor 488 Goat anti-Rabbit IgG (invitrogen, A11008) diluted 500-fold with Blocking solution at room temperature for 45 minutes, and washed with PBS(−) three times, and treated with 4% paraformaldehyde/PBS(−) at room temperature for 5 minutes, and washed with PBS(−) twice. Thereafter, it was treated with 70%, 95% and 100% EtOH for 3, 2 and 2 minutes in series, respectively. After air drying, it was reacted with Cy-3-labeled 3′-(CCCTAA)₄-5′ probe (PANAGENE Inc., F1002) at 80° C. for 3 minutes. Thereafter, it was washed with a washing solution (70% formamide, 10 mM Tris-HCl (pH 7.2)), washed with PBS(−) three times, then, reacted with 0.25 μg/mL DAPI at room temperature for 5 minutes, and further, washed with PBS(−) 3 times. After enclosing the slide glass, it was observed with a fluorescence microscope (Zwiss, Axiovert 200M). 53BP1 (TIF) localized in telomere was measured using an image analysis software Columbus. Those having 4 or more TIF per nucleus were taken as TIF-positive cells. The result is shown in FIG. 5. FIG. 5(a) is a photograph of cells obtained by using a fluorescence microscope, and the arrow indicates the portion where colocalization of telomere and 53BP1 is seen. FIG. 5(b) is a graph showing the result obtained by counting the number of cells in which colocalization has occurred, that is, TIF has occurred, using a fluorescence microscope, and calculating the ratio thereof.

As shown in FIG. 5, in the control, almost no colocalization of telomere with 53BP1 was observed, but in the cells treated with the compound #198, colocalization of telomere with 53BP1 was observed. That is, it is considered that TIF is generated and telomere has abnormality in the cells treated with the compound #198. This is believed to be ascribable to the fact that the compound #198 inhibits TRF2 from binding to telomere.

As mentioned above, the compound #198 is believed to promote telomere abnormality. Here, since telomere abnormality is known to be associated with cell aging and cell death, whether the compound #198 causes apoptosis of cells or not was examined utilizing FACS. The method and the result are described below.

HeLa1.2.11 cells were treated with 20 μM of a compound #198 or DMSO for 48 hours, then, the cells were collected together with the supernatant in a 15 mL tube and centrifuged at 1000 rpm for 3 minutes, to remove the supernatant. The cells were resuspended in 5 ml of PBS(−) and centrifuged at 1000 rpm for 3 minutes, to remove the supernatant. The cells were resuspended with 500 μl of 1×Binding buffer (MBL, 4695-300), and 90 μl of which was transferred to a 5 mL tube (BD Falcon, 352052), and 10 μl of Annexin V-FITC (MBL, 4700-100) was added to stain. Incubation was carried out for 15 minutes while protected from light, and analysis was performed. Annexin V-positive cells were detected by a Cell sorter (SONY, SH-800). The result is shown in FIG. 6.

As shown in FIG. 6, many cells were Annexin V positive in the case where treated with a compound #198 as compared to the control treated with DMSO. That is, this suggests that the compound #198 induces cell apoptosis.

Furthermore, whether the expression of cleaved caspase-3 which is known to be a crucial factor of apoptosis is promoted by a compound #198 or not was analyzed by Western blotting, to demonstrate that the compound #198 induces cell apoptosis, by another method. The method and the result are described below.

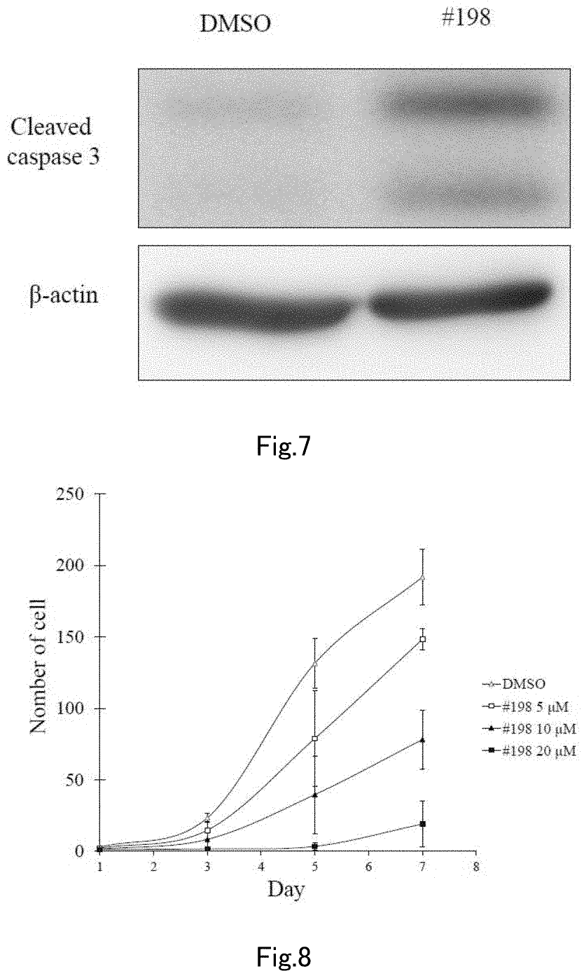

HeLa1.2.11 cells were treated with 20 μM of a compound #198 or DMSO for 48 hours, then, the cells were collected together with the supernatant in a 15 mL tube and centrifuged at 1000 rpm for 3 minutes, to remove the supernatant. The cell pellet was dissolved with 2×Sample buffer (117 mM Tris-HCl (pH 6.8), 13% glycerol, 3.7% SDS, 200 mM DTT, 0.004% bromo phenol blue) and thermally denatured at 95° C. for 5 minutes. A 10 μg sample was electrophoresed on 8% acrylamide gel in Running buffer (25 mM Tris, 192 mM glycine, 0.1% (w/v) SDS) at a constant voltage (120 V). Thereafter, it was transferred to a PVDF membrane filter Immobilon-P (MILLIPORE), and the intended protein was detected using an antigen-antibody reaction. As the antibodies, B-actin (SIGMA, A5441) or Cleared caspase-3 (CST, D175) was used as the primary antibody, and Peroxidase-labeled Goat anti-mouse/anti-rabbit secondary antibody (Jackson Immuno Research, 111-035-003/115-035-003) was used as the secondary antibody, and the signal was detected using Luminoanalyzer (ImageQuant, LAS4000). The result is shown in FIG. 7.

As shown in FIG. 7, in the control treated with DMSO, cleaved caspase-3 was hardly observed, while in the cells treated with a compound #198, cleaved caspase-3 was detected. It is considered from this result that the compound #198 promotes production of cleaved caspase-3, that is, induces cell apoptosis.

Next, the influence of a compound #198 on proliferation of cells was examined. The method and the result are shown below.

HeLa1.2.11 cells were seeded at 1×10⁴ cells/35 mm dish, and on the next day, treated with a compound #198 (5 μM, 10 μM, 20 μM) or DMSO as a control (treatment day is day 1), and the cells were counted on days 1, 3, 5 and 7. The result is shown in FIG. 8.

As shown in FIG. 8, proliferation of cells was suppressed depending on the treatment concentration with the compound #198.

Further, the effect of suppressing proliferation of HeLa1.2.11 cells by each of the above-described candidate compounds other than the compound #198 was measured in the same manner as described above, and the IC50 was calculated. The results are shown in Table 2 below.

TABLE 2

| candidate compound | IC50 |
|---|---|
| #144 | 31 μM |
| #145 | 26 μM |
| #151 | 17 μM |
| #153 | 34 μM |
| #168 | 43 μM |
| #171 | 48 μM |
| #192 | 24 μM |
| #198 | 22 μM |
| #201 | 15 μM |
| #204 | 20 μM |
| #207 | 19 μM |
| #224 | 12 μM |
| #225 | 28 μM |
| #226 | 34 μM |
| #227 | 18 μM |
| #228 | 20 μM |

As shown in Table 2, it is clear that the candidate compounds other than the compound #198 also perform the effect of suppressing proliferation of cells.

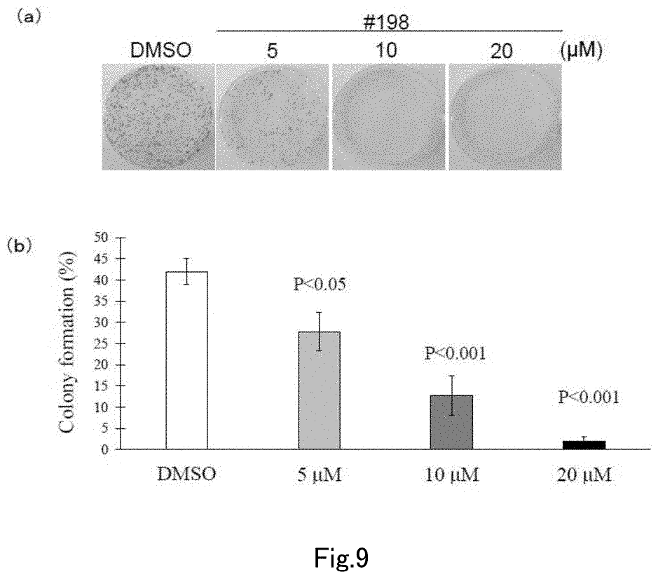

Next, HeLa 1.2.11 cells were seeded at 1×10³ cells/100 mm dish, and on the next day, treated with a compound #198 (5 to 20 μM) or DMSO as a control, and 10 days after seeding, the formed colony was fixed with 100% EtOH, then, stained with 4% Giemsa stain (Muto Pure Chemicals Co., Ltd., 1500-2)/PBS(−) and measured. The result is shown in FIG. 9. FIG. 9(a) shows a photograph of the Giemsa-stained plate described above, and FIG. 9(b) is a graph showing the result obtained by calculating the generation rate of the colony.

As shown in FIG. 9, generation of the colony was suppressed depending on the treatment concentration of the compound #198.

It is considered from the results of the above-described examples that the compound according to the present invention inhibits a telomere-binding protein from binding to telomere, and resultantly, inhibits G-tail formation in telomere, to shorten the G-tail. According to this, the compound according to the present invention is considered to cause suppression of proliferation of cells and induction of apoptosis. Hence, the compound according to the present invention may possibly be used as a reagent for inducing cell aging or cell death, and further, may possibly be applied to the development of therapeutic agents for various diseases such as cancers.

### [Sequence List]

1. A compound represented by the following chemical formula: 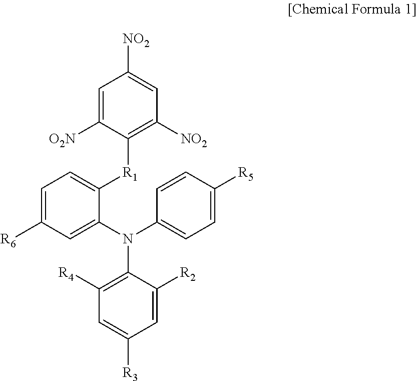
wherein, in the above-described chemical formula,
R₁ is oxygen, and
R₂ to R₆ are each independently selected from hydrogen, an alkyl group having 1 to 6 carbon atoms, an alkoxy group having 1 to 6 carbon atoms, an acyl group having 1 to 6 carbon atoms and a nitro group.

2. The compound according to claim 1,
wherein, in the above-described chemical formula,
R₁ is oxygen,
R₂ and R₄ are each independently hydrogen or a nitro group,
R₃ is hydrogen, a nitro group, a methyl group, a methoxy group or a butyl group,
R₅ is hydrogen, a methyl group, a methoxy group or an acetyl group, and
R₆ is hydrogen or a butyl group.

3. The compound according to claim 2, represented by any one of the following chemical formulae:

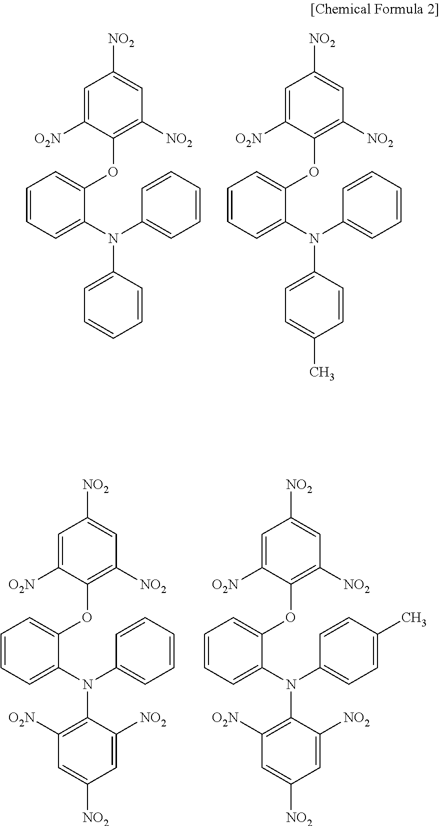

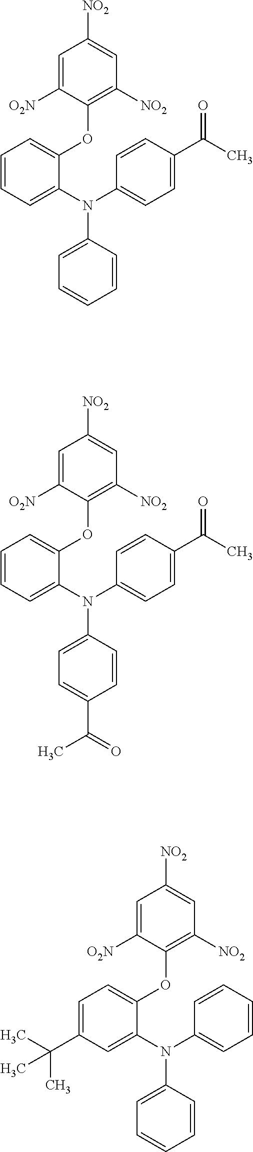

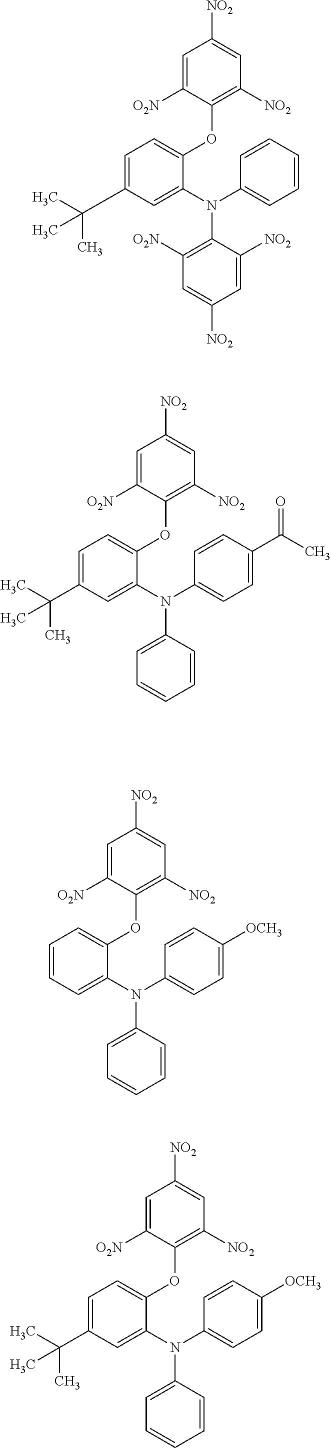

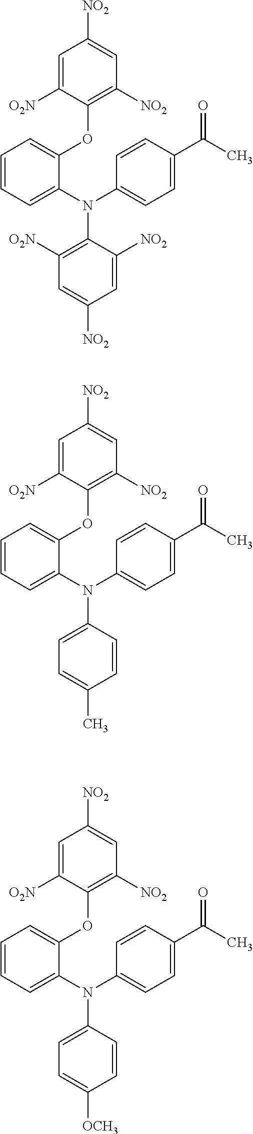 [SMILES: CC(=O)c1ccc(N(c2ccccc2Oc2c([N+](=O)[O-])cc([N+](=O)[O-])cc2[N+](=O)[O-])c2c([N+](=O)[O-])cc([N+](=O)[O-])cc2[N+](=O)[O-])cc1 ; CC(=O)c1ccc(N(c2ccc(C)cc2)c2ccccc2Oc2c([N+](=O)[O-])cc([N+](=O)[O-])cc2[N+](=O)[O-])cc1 ; COc1ccc(N(c2ccc(C(C)=O)cc2)c2ccccc2Oc2c([N+](=O)[O-])cc([N+](=O)[O-])cc2[N+](=O)[O-])cc1]

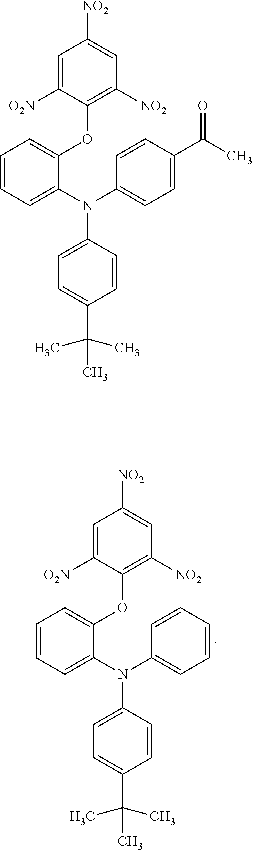 [SMILES: CC(=O)c1ccc(N(c2ccc(C(C)(C)C)cc2)c2ccccc2Oc2c([N+](=O)[O-])cc([N+](=O)[O-])cc2[N+](=O)[O-])cc1 ; CC(C)(C)c1ccc(N(c2ccccc2)c2ccccc2Oc2c([N+](=O)[O-])cc([N+](=O)[O-])cc2[N+](=O)[O-])cc1]

4. A method for inhibiting the telomere-binding protein from binding to telomere DNA, the method comprising contacting a cell or a sample containing the telomere-binding protein and telomere DNA with a compound represented by the following chemical formula: 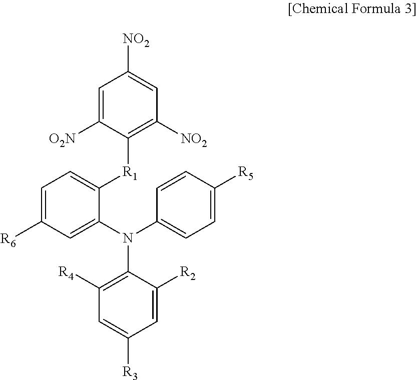
wherein, in the above-described chemical formula,
R₁ is oxygen or sulfur, and
R₂ to R₆ are each independently selected from hydrogen, an alkyl group having 1 to 6 carbon atoms, an alkoxy group having 1 to 6 carbon atoms, an acyl group having 1 to 6 carbon atoms and a nitro group.

5. The method according to claim 4,
wherein, in the above-described chemical formula,
R₁ is oxygen or sulfur,
R₂ and R₄ are each independently hydrogen or a nitro group,
R₃ is hydrogen, a nitro group, a methyl group, a methoxy group or a butyl group,
R₅ is hydrogen, a methyl group, a methoxy group or an acetyl group, and
R₆ is hydrogen or a butyl group.

6. The method according to claim 4, comprising a compound represented by any one of the following chemical formulae:

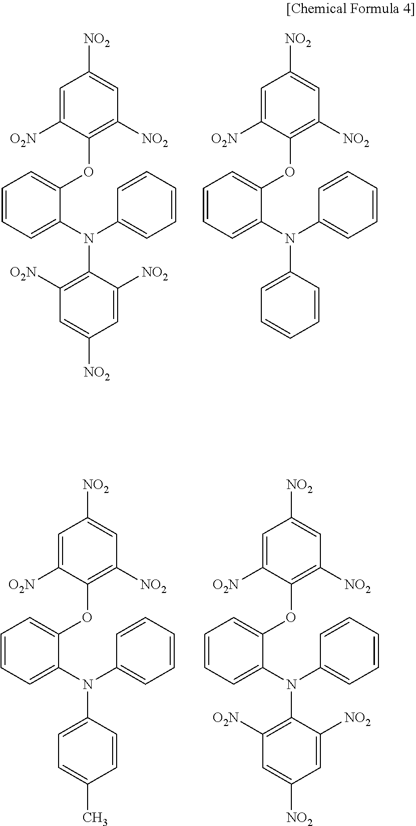

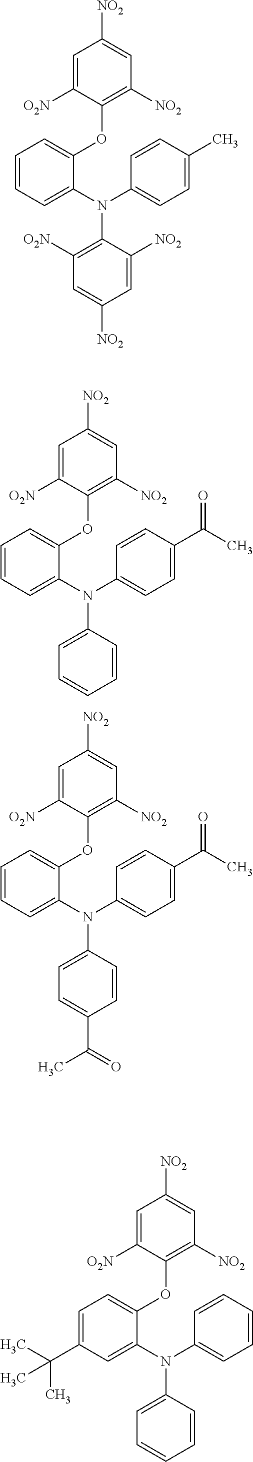

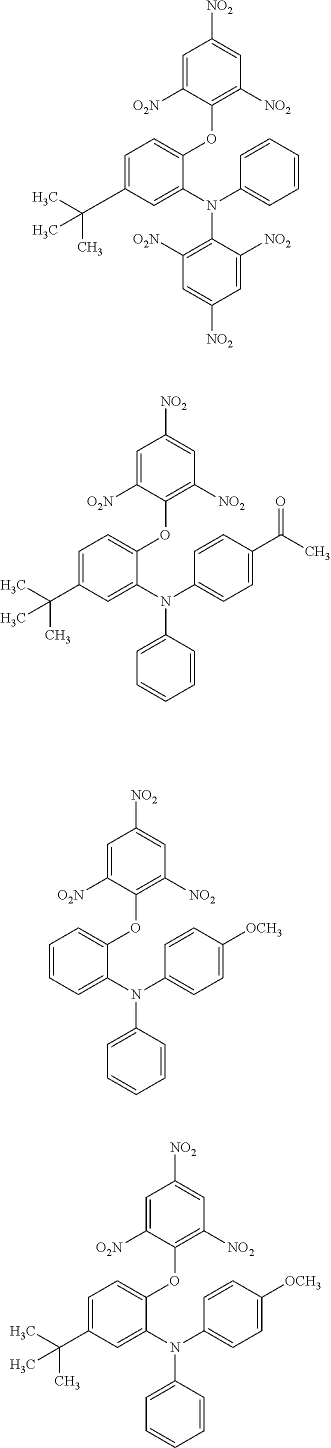

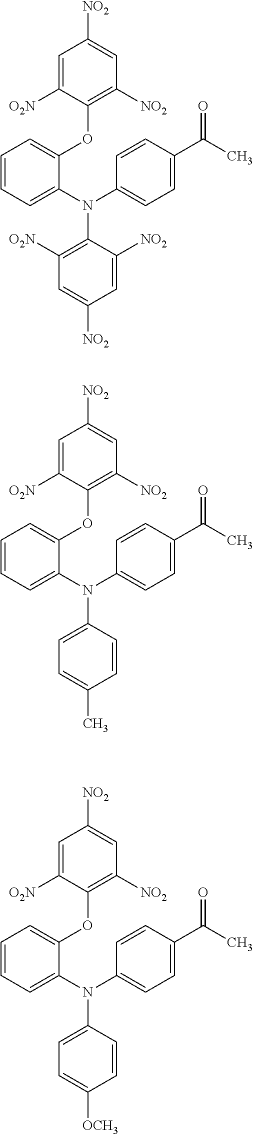 [SMILES: CC(=O)c1ccc(N(c2ccccc2Oc2c([N+](=O)[O-])cc([N+](=O)[O-])cc2[N+](=O)[O-])c2c([N+](=O)[O-])cc([N+](=O)[O-])cc2[N+](=O)[O-])cc1 ; CC(=O)c1ccc(N(c2ccc(C)cc2)c2ccccc2Oc2c([N+](=O)[O-])cc([N+](=O)[O-])cc2[N+](=O)[O-])cc1 ; COc1ccc(N(c2ccc(C(C)=O)cc2)c2ccccc2Oc2c([N+](=O)[O-])cc([N+](=O)[O-])cc2[N+](=O)[O-])cc1]

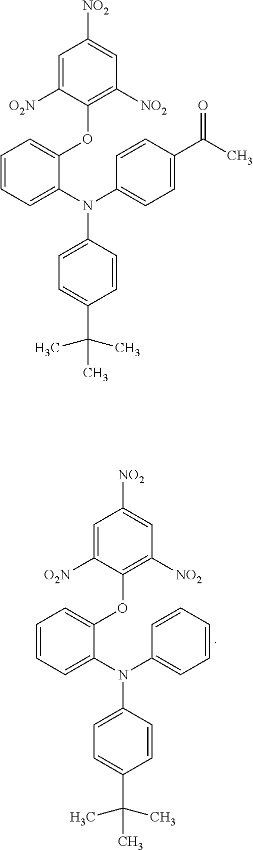 [SMILES: CC(=O)c1ccc(N(c2ccc(C(C)(C)C)cc2)c2ccccc2Oc2c([N+](=O)[O-])cc([N+](=O)[O-])cc2[N+](=O)[O-])cc1 ; CC(C)(C)c1ccc(N(c2ccccc2)c2ccccc2Oc2c([N+](=O)[O-])cc([N+](=O)[O-])cc2[N+](=O)[O-])cc1]

7. The method according to claim 4, wherein the above-described telomere-binding protein is TRF1, TRF2, or POT1.

8.-13. (canceled)

14. A method for treating or preventing cancers, comprising administering a compound represented by the following chemical formula to a patient: 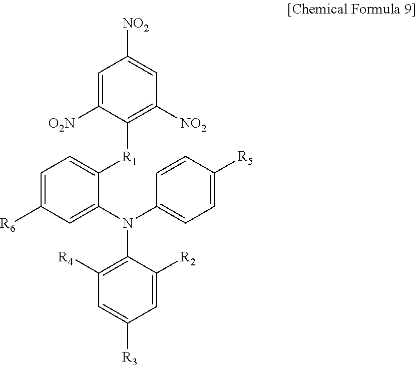
wherein, in the above-described chemical formula,
R₁ is oxygen or sulfur, and
R₂ to R₆ are each independently selected from hydrogen, an alkyl group having 1 to 6 carbon atoms, an alkoxy group having 1 to 6 carbon atoms, an acyl group having 1 to 6 carbon atoms and a nitro group.

15. The method according to claim 14, wherein, in the above-described chemical formula,
R₁ is oxygen or sulfur,
R₂ and R₄ are each independently hydrogen or a nitro group,
R₃ is hydrogen, a nitro group, a methyl group, a methoxy group or a butyl group,
R₅ is hydrogen, a methyl group, a methoxy group or an acetyl group, and
R₆ is hydrogen or butyl group.

16. The method according to claim 14, wherein the above-described compound is a compound represented by any one of the following chemical formulae: 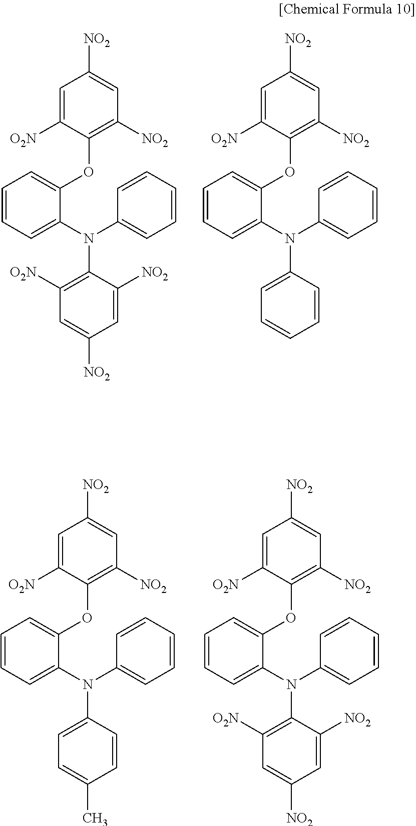 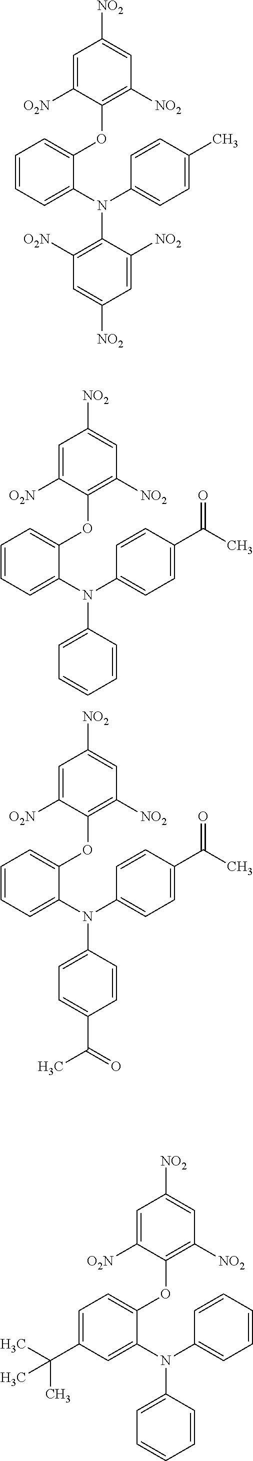 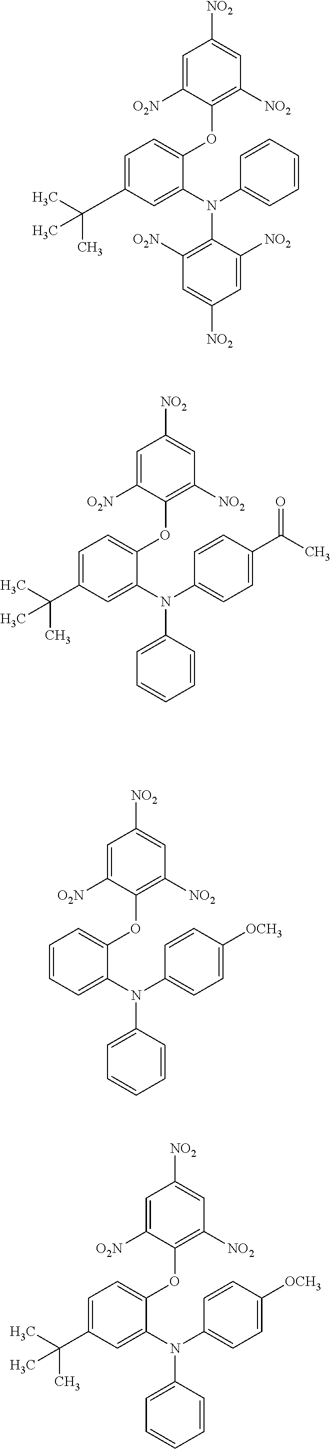 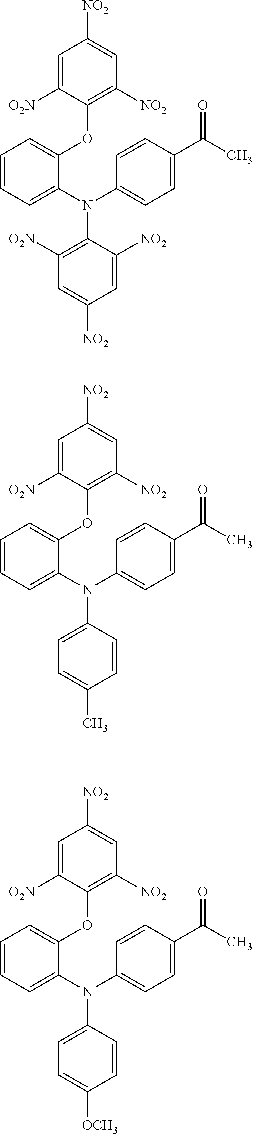 [SMILES: CC(=O)c1ccc(N(c2ccccc2Oc2c([N+](=O)[O-])cc([N+](=O)[O-])cc2[N+](=O)[O-])c2c([N+](=O)[O-])cc([N+](=O)[O-])cc2[N+](=O)[O-])cc1 ; CC(=O)c1ccc(N(c2ccc(C)cc2)c2ccccc2Oc2c([N+](=O)[O-])cc([N+](=O)[O-])cc2[N+](=O)[O-])cc1 ; COc1ccc(N(c2ccc(C(C)=O)cc2)c2ccccc2Oc2c([N+](=O)[O-])cc([N+](=O)[O-])cc2[N+](=O)[O-])cc1] 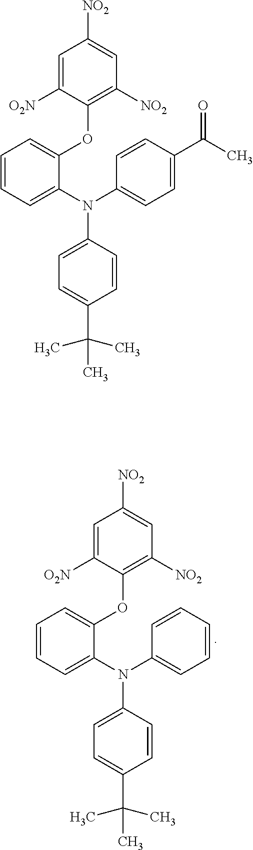 [SMILES: CC(=O)c1ccc(N(c2ccc(C(C)(C)C)cc2)c2ccccc2Oc2c([N+](=O)[O-])cc([N+](=O)[O-])cc2[N+](=O)[O-])cc1 ; CC(C)(C)c1ccc(N(c2ccccc2)c2ccccc2Oc2c([N+](=O)[O-])cc([N+](=O)[O-])cc2[N+](=O)[O-])cc1]
